SEQUENCE LISTING (40 sequences)

SEQ ID NO: 1 - DNA, 19 bp, Artificial Sequence
  GAPDH primer- Forward Sequence
aacctgccaagtatgatga

SEQ ID NO: 2 - DNA, 18 bp, Artificial Sequence
  GAPDH primer- Reverse Sequence
ggagttgctgttgaagtc

SEQ ID NO: 3 - DNA, 20 bp, Artificial Sequence
  LXR-L\ primer- Forward Sequence
gctctgctcatagccatcag

SEQ ID NO: 4 - DNA, 20 bp, Artificial Sequence
  LXR-L\ primer- Reverse Sequence
cagggcctccacatatgtgt

SEQ ID NO: 5 - DNA, 20 bp, Artificial Sequence
  SREBP-1c primer- Forward Sequence
cacagcggttttgaacgaca

SEQ ID NO: 6 - DNA, 20 bp, Artificial Sequence
  SREBP-1c primer- Reverse Sequence
ctctcaggagagttggcacc

SEQ ID NO: 7 - DNA, 21 bp, Artificial Sequence
  ACC primer- Forward Sequence
ggaggctgcattgaacacaag

SEQ ID NO: 8 - DNA, 20 bp, Artificial Sequence
  ACC primer- Reverse Sequence
cgacggtgaaatctctgtgc

SEQ ID NO: 9 - DNA, 21 bp, Artificial Sequence
  FAS primer- Forward Sequence
gctgcggaaacttcaggaaat

SEQ ID NO: 10 - DNA, 23 bp, Artificial Sequence
  FAS primer- Reverse Sequence
agagacgtgtcactcctggactt

SEQ ID NO: 11 - DNA, 23 bp, Artificial Sequence
  ME primer- Forward Sequence
aactctgacttcgacaggtatct

SEQ ID NO: 12 - DNA, 21 bp, Artificial Sequence
  ME primer- Reverse Sequence
cggaatgccaaactgtactgc

SEQ ID NO: 13 - DNA, 21 bp, Artificial Sequence
  PPAR-L\ primer- Forward Sequence
tgacaccttcctcttcccaaa

SEQ ID NO: 14 - DNA, 20 bp, Artificial Sequence
  PPAR-L\ primer- Reverse Sequence
cgtcggactcggtcttcttg

SEQ ID NO: 15 - DNA, 20 bp, Artificial Sequence
  RXR-L\ primer- Forward Sequence
ccaaacatttcctgccgctc

SEQ ID NO: 16 - DNA, 20 bp, Artificial Sequence
  RXR-L\ primer- Reverse Sequence
cgacccgttggagagttgag

SEQ ID NO: 17 - DNA, 20 bp, Artificial Sequence
  CPT1 primer- Forward Sequence
ctgagccatgaagccctcaa

SEQ ID NO: 18 - DNA, 20 bp, Artificial Sequence
  CPT1 primer- Reverse Sequence
cacacccaccaccacgataa

SEQ ID NO: 19 - DNA, 20 bp, Artificial Sequence
  UCP2 primer- Forward Sequence
acaagaccattgcacgagag

SEQ ID NO: 20 - DNA, 20 bp, Artificial Sequence
  UCP2 primer- Reverse Sequence
atgaggttggctttcaggag

SEQ ID NO: 21 - DNA, 21 bp, Artificial Sequence
  TLR4 primer- Forward Sequence
accaggaagcttgaatccctg

SEQ ID NO: 22 - DNA, 22 bp, Artificial Sequence
  TLR4 primer- Reverse Sequence
tcatcagggactttgctgagtt

SEQ ID NO: 23 - DNA, 22 bp, Artificial Sequence
  MyD88  primer- Forward Sequence
catggtggtggttgtttctgac

SEQ ID NO: 24 - DNA, 21 bp, Artificial Sequence
  MyD88 primer- Reverse Sequence
ctggagacaggctgagtgcaa

SEQ ID NO: 25 - DNA, 20 bp, Artificial Sequence
  NF-LeB primer- Forward Sequence
ccgtgtttgttcagcttcgg

SEQ ID NO: 26 - DNA, 20 bp, Artificial Sequence
  NF-LeB primer- Reverse Sequence
ctgtccgagaagttcggcat

SEQ ID NO: 27 - DNA, 20 bp, Artificial Sequence
  iNOS primer- Forward Sequence
ggcagcctgtgagacctttg

SEQ ID NO: 28 - DNA, 21 bp, Artificial Sequence
  iNOS primer- Reverse Sequence
gcattggaagtgaagcgtttc

SEQ ID NO: 29 - DNA, 19 bp, Artificial Sequence
  COX-2 primer- Forward Sequence
tgggttcacccgaggactg

SEQ ID NO: 30 - DNA, 21 bp, Artificial Sequence
  COX-2 primer- Reverse Sequence
ggggatacacctctccaccaa

SEQ ID NO: 31 - DNA, 25 bp, Artificial Sequence
  L\-SMA primer- Forward Sequence
ttcgtgactactgccgagcgtgaga

SEQ ID NO: 32 - DNA, 17 bp, Artificial Sequence
  L\-SMA primer- Reverse Sequence
aaagatggctggaagag

SEQ ID NO: 33 - DNA, 17 bp, Artificial Sequence
  ADH primer- Forward Sequence
ggccgccttgacaccat

SEQ ID NO: 34 - DNA, 21 bp, Artificial Sequence
  ADH primer- Reverse Sequence
gcactcctacgacgacgctta

SEQ ID NO: 35 - DNA, 22 bp, Artificial Sequence
  ALDH primer- Forward Sequence
cgaacgtctgccctatcaactt

SEQ ID NO: 36 - DNA, 19 bp, Artificial Sequence
  ALDH primer- Reverse Sequence
ccggaatcgaaccctgatt

SEQ ID NO: 37 - DNA, 17 bp, Artificial Sequence
  CYP2E1 primer- Forward Sequence
gcccgcatccaaagaga

SEQ ID NO: 38 - DNA, 19 bp, Artificial Sequence
  CYP2E1 primer- Reverse Sequence
ggctggcctttggtctttt

SEQ ID NO: 39 - DNA, 22 bp, Artificial Sequence
  CAT primer- Forward Sequence
tgagaagcctaagaacgcaatt

SEQ ID NO: 40 - DNA, 17 bp, Artificial Sequence
  CAT primer- Reverse Sequence
cccttcgcagccatgtg

(19) United States
(12) Patent Application Publication
(10) Pub. No.: US 2017/0216315 A1
(43) Pub. Date: Aug. 3, 2017

(54) USES OF ERGOSTATRIEN-3beta-OL

(71) Applicants: CHINA MEDICAL UNIVERSITY, Taichung City (TW); NATIONAL TAIWAN UNIVERSITY, Taipei City (TW)

(72) Inventors: Yueh-Hsiung KUO, Taipei City (TW); Yi-Chen CHEN, Taipei City (TW); Yi-Chen LIU, Kaohsiung City (TW)

(73) Assignees: CHINA MEDICAL UNIVERSITY, Taichung City (TW); NATIONAL TAIWAN UNIVERSITY, Taipei City (TW)

(21) Appl. No.: 15/199,065

(22) Filed: Jun. 30, 2016

(30) Foreign Application Priority Data
Feb. 3, 2016 (TW) 105103479

(51) Int. Cl. A61K 31/575

(52) U.S. Cl. CPC A61K 31/575

(57) ABSTRACT

A use of an active ingredient in the manufacture of a medicament or a preparation is provided, wherein the active ingredient is at least one of ergstatrien-3β-ol and a pharmaceutically acceptable ester thereof, the medicament is for alleviating, inhibiting and/or treating the hepatic injuries caused by alcohol consumption, or for alleviating and/or inhibiting body fat accumulation caused by alcohol consumption, and the preparation is a food or a food additive for increasing the alcohol metabolism capability of the liver.

### CROSS REFERENCE TO RELATED APPLICATIONS

This application claims priority to Taiwan Patent Application No. 105103479 filed on Feb. 3, 2016, in the Taiwan Intellectual Property Office, the disclosure of which is incorporated herein in its entirety by reference.

### FIELD OF THE INVENTION

The present invention relates to the use of ergostatrien-3β-ol for alleviating, inhibiting and/or treating hepatic injuries caused by alcohol consumption, alleviating and/or inhibiting body fat accumulation caused by alcohol consumption, and/or increasing the alcohol metabolism capability of liver.

### BACKGROUND OF THE INVENTION

“Drinking” is a common phenomenon in social gatherings and celebrations around the world. However, research has shown that alcohol consumption increases the lipogenesis in the human body and decreases the metabolism and transportation of fatty acid, and thus, causes body fat accumulation and leads to various fatty acid metabolism-related diseases, such as hyperlipidemia, arteriosclerotic cardiovascular disease, cardiac arrhythmia, heart failure, vascular obstruction, fatty liver, etc. Fatty liver is the most common of the above diseases, and it could further develop into hepatitis, or even convert into liver fibrosis, liver cirrhosis, or hepatic carcinoma.

In addition, it has been known that excessive alcohol consumption relates to the occurrences of various diseases. For example, excessive alcohol consumption may induce alcoholic cardiomyopathy (ACM) and coronary artery disease (CAD), directly affect the function and construct of kidney and change its capability of modulating the volume, composition, and electrolyte ratio of body fluid, cause central nervous system (CNS) degeneration and cerebral dysfunction, and cause the deterioration of liver disease, etc. Relevant description can be seen in articles, such as “Alcohol and lipid metabolism. Am J Physiol Endocrinol Metab. 295: 10-16 (2008);” “Alcohol abuse and heart failure. European Journal of Heart Failure. 11: 453-462 (2009);” “Alcohol's impact on kidney function. Alcohol Health Res world. 21(1): 84-92 (1997);” and “DNA damage and neurotoxicity of chronic alcohol abuse. Experimental Biology and Medicine. 237(7): 740-747 (2012)”, which are incorporated herein by reference.

Because of the above adverse effects on the human body caused by alcohol, there are many commercially available health foods being alleged to protect the liver under chronic alcohol consumption which is a type of regular alcohol consumption. However, most of these health foods are only directed to the improvement of the antioxidant function of liver, and are not effective in modulating the body lipid homeostasis or in increasing the alcohol metabolism capability of liver. Because of the strong business entertainment culture, and other social situations in which drinking is unavoidable, if a medicament or preparation with more efficiency in increasing the alcohol metabolism capability of liver, modulating the lipid homeostasis, and/or anti-inflammation can be developed, it will be favorable for decreasing incidence of many diseases caused by alcohol consumption.

The inventors of the present invention found that ergostatrien-3β-ol is effective in increasing the alcohol metabolism capability of liver. Therefore, ergostatrien-3β-ol can be used to alleviate or avoid the harm caused by alcohol consumption. In addition, the inventors also found that for a subject with body fat accumulation or hepatic injuries caused by alcohol consumption, ergostatrien-3β-ol is capable of effectively alleviating and/or inhibiting the body fat accumulation, or alleviating, inhibiting and/or treating the hepatic injuries, wherein the hepatic injuries include hepatitis, liver fibrosis, liver cirrhosis, hepatic carcinoma, etc.

Therefore, with the use of one single active ingredient (i.e., ergostatrien-3β-ol), the present invention can achieve one or more of the following effects at a relatively lower manufacturing cost: alleviating, inhibiting and/or treating hepatic injuries caused by alcohol consumption, alleviating and/or inhibiting body fat accumulation caused by alcohol consumption, and increasing the alcohol metabolism capability of the liver.

### SUMMARY OF THE INVENTION

An objective of the present invention is to provide a use of an active ingredient in the manufacture of a medicament, wherein the active ingredient is at least one of ergostatrien-3β-ol and a pharmaceutically acceptable ester of ergostatrien-3β-ol, and the medicament is used for alleviating, inhibiting and/or treating hepatic injuries caused by alcohol consumption, especially for alleviating, inhibiting and/or treating hepatic injuries caused by chronic alcohol consumption.

Another objective of the present invention is to provide a use of an active ingredient in the manufacture of a medicament, wherein the active ingredient is at least one of ergostatrien-3β-ol and a pharmaceutically acceptable ester of ergostatrien-3β-ol, and the medicament is used for alleviating and/or inhibiting body fat accumulation caused by alcohol consumption, especially for alleviating and/or inhibiting body fat accumulation caused by chronic alcohol consumption.

Still another objective of the present invention is to provide a use of an active ingredient in the manufacture of a preparation, wherein the active ingredient is at least one of ergostatrien-3β-ol and a pharmaceutically acceptable ester of ergostatrien-3β-ol, and the preparation is a food or a food additive used for increasing the alcohol metabolism capability of the liver.

Yet another objective of the present invention is to provide a method of alleviating, inhibiting and/or treating hepatic injuries caused by alcohol consumption, comprising administering to a subject in need an effective amount of an active ingredient selected from the group consisting of ergostatrien-3β-ol, a pharmaceutically acceptable ester of ergostatrien-3β-ol, and combinations thereof. The present invention is especially to provide a method of alleviating, inhibiting, and/or treating hepatic injuries caused by chronic alcohol consumption.

Yet another objective of the present invention is to provide a method of alleviating and/or inhibiting body fat accumulation caused by alcohol consumption, comprising administering to a subject in need an effective amount of an active ingredient selected from the group consisting of ergostatrien-3β-ol, a pharmaceutically acceptable ester of ergostatrien-3β-ol, and combinations thereof. The present invention is especially to provide a method of alleviating and/or inhibiting body fat accumulation caused by chronic alcohol consumption.

Yet another objective of the present invention is to provide a method of increasing the alcohol metabolism capability of the liver, comprising administering to a subject in need an effective amount of an active ingredient selected from the group consisting of ergostatrien-3β-ol, a pharmaceutically acceptable ester of ergostatrien-3β-ol, and combinations thereof.

The detailed technology and preferred embodiments implemented for the present invention are described in the following paragraphs accompanying the appended drawings for people skilled in this field to well appreciate the features of the claimed inventive.

### BRIEF DESCRIPTION OF THE DRAWINGS

The patent application contains at least one drawing executed in color. Copies of this patent document with color drawing(s) will be provided by the Patent and Trademark Office upon request and payment of the necessary fee.

FIG. 1 is a body weight-change curve showing the effects of alcohol consumption and ergostatrien-3β-ol (hereinafter referred to as “EK100”) intake on mice body weight. The curve shows the changes of mice body weight during the experiment (4 weeks), wherein the control group (referred to as “control”) was fed daily with a normal liquid diet, the alcohol-treated group (referred to as “EtOH”) was fed daily with a Lieber-DeCarli alcoholic liquid diet, the 1× ergostatrien-3β-ol group (referred to as “EK100_1X”) was fed daily with a Lieber-DeCarli alcoholic liquid diet and gavage fed with 1 mg/kg of ergostatrien-3β-ol daily, the 5X ergostatrien-3β-ol group (referred to as “EK100_5X”) was fed daily with a Lieber-DeCarli alcoholic liquid diet and gavage fed with 5 mg/kg of ergostatrien-3β-ol daily, and the 10X ergostatrien-3β-ol group (referred to as “EK100_10X”) was fed daily with a Lieber-DeCarli alcoholic liquid diet and gavage fed with 10 mg/kg of ergostatrien-3β-ol daily.

FIGS. 2A, 2B and 2C show the effects of alcohol consumption and ergostatrien-3β-ol intake on the amounts of triacylglycerol (TAG) and total cholesterol (TC) in the mice body, wherein, FIG. 2A is a bar diagram showing the amounts of TAG and TC in the serum, FIG. 2B is a bar diagram showing the amounts of TAG and TC in the liver, and FIG. 2C is a bar diagram showing the amounts of TAG and TC in the feces. FIGS. 2A, 2B, and 2C all comprise the results of the control group (referred to as “control”), alcohol-treated group (referred to as “EtOH”), 1X ergostatrien-3β-ol group (referred to as “EK100_1X”), 5X ergostatrien-3β-ol group (referred to as “EK100_5X”), and 10X ergostatrien-3β-ol group (referred to as “EK100_10X”).

FIG. 3 is a bar diagram showing the effects of alcohol consumption and ergostatrien-3β-ol intake on the amount of bile acid excreted by the mice, wherein the results of the amount of bile acid in the feces of the control group (referred to as “control”), alcohol-treated group (referred to as “EtOH”), 1X ergostatrien-3β-ol group (referred to as “EK100_1X”), 5X ergostatrien-3β-ol group (referred to as “EK100_5X”), and 10X ergostatrien-3β-ol group (referred to as “EK100_10X”) are shown.

FIG. 4 is a bar diagram showing the effects of alcohol consumption and ergostatrien-3β-ol intake on the expressions of lipid synthesis-related genes (i.e., LXR-α, SREBP-1c, ACC, FAS, and ME) in the livers of the mice, wherein the results of the control group (referred to as “control”), alcohol-treated group (referred to as “EtOH”), 1X ergostatrien-3β-ol group (referred to as “EK100_1X”), 5X ergostatrien-3β-ol group (referred to as “EK100_5X”), and 10X ergostatrien-3β-ol group (referred to as “EK100_10X”) are shown.

FIG. 5 is a bar diagram showing the effects of alcohol consumption and ergostatrien-3β-ol intake on the expressions of fatty acid β-oxidation-promoting genes (i.e., PPAR-α, RXR-α, CPT1, and UCP2) in the livers of the mice, wherein the results of the control group (referred to as “control”), alcohol-treated group (referred to as “EtOH”), 1X ergostatrien-3β-ol group (referred to as “EK100_1X”), 5X ergostatrien-3β-ol group (referred to as “EK100_5X”), and 10X ergostatrien-3β-ol group (referred to as “EK100_10X”) are shown.

FIGS. 6A to 6E are photographs showing the appearance of the livers of the mice that consumed alcohol and took ergostatrien-3β-ol, wherein each of FIGS. 6A, 6B, 6C, 6D, and 6E shows the appearance of the livers of the control group (referred to as “control”), alcohol-treated group (referred to as “EtOH”), 1X ergostatrien-3β-ol group (referred to as “EK100_1X”), 5X ergostatrien-3β-ol group (referred to as “EK100_5X”), and 10X ergostatrien-3β-ol group (referred to as “EK100_10X”) respectively.

FIGS. 7A to 7E are photographs showing fat accumulation in the livers of the mice that consumed alcohol and took ergostatrien-3β-ol, wherein each of FIGS. 7A, 7B, 7C, 7D, and 7E shows the Hematoxylin-eosin (H&E) staining result of the liver of the control group (referred to as “control”), alcohol-treated group (referred to as “EtOH”), 1X ergostatrien-3β-ol group (referred to as “EK100_1X”), 5X ergostatrien-3β-ol group (referred to as “EK100_5X”), and 10X ergostatrien-3β-ol group (referred to as “EK100_10X”) respectively.

FIG. 8 is a bar diagram showing the effects of alcohol consumption and ergostatrien-3β-ol intake on the activities of aspartate aminotransferase (AST) and alanine aminotransferase (ALT) in the serum of the mice, wherein the results of control group (referred to as “control”), alcohol-treated group (referred to as “EtOH”), 1X ergostatrien-3β-ol group (referred to as “EK100_1X”), 5X ergostatrien-3β-ol group (referred to as “EK100_5X”), and 10X ergostatrien-3β-ol group (referred to as “EK100_10X”) are shown.

FIGS. 9A and 9B show the effects of alcohol consumption and ergostatrien-3β-ol intake on the concentrations of TNF-α and IL-1β in the livers of the mice, wherein FIG. 9A is a bar diagram showing the concentrations of TNF-α, and FIG. 9B is a bar diagram showing the concentrations of IL-1β. FIGS. 9A and 9B both include the results of the control group (referred to as “control”), alcohol-treated group (referred to as “EtOH”), 1X ergostatrien-3β-ol group (referred to as “EK100_1X”), 5X ergostatrien-3β-ol group (referred to as “EK100_5X”), and 10X ergostatrien-3β-ol group (referred to as “EK100_10X”).

FIG. 10 is a bar diagram showing the effects of alcohol consumption and ergostatrien-3β-ol intake on the expressions of inflammation-promoting genes (i.e., TLR4, MyD88, NF-κB, iNOS, COX-2, and α-SMA) in the livers of the mice, wherein the results of the control group (referred to as “control”), alcohol-treated group (referred to as “EtOH”), 1X ergostatrien-3β-ol group (referred to as “EK100_1X”), 5X ergostatrien-3β-ol group (referred to as “EK100_5X”), and 10X ergostatrien-3β-ol group (referred to as “EK100_10X”) are shown.

FIG. 11 is a bar diagram showing the results of liver inflammation of the mice that consumed alcohol and took ergostatrien-3β-ol, wherein the HAI scores of portal inflammation, lobular inflammation, and periportal necrosis in the livers of the control group (referred to as “control”), alcohol-treated group (referred to as “EtOH”), 1X ergostatrien-3β-ol group (referred to as “EK100_1X”), 5X ergostatrien-3β-ol group (referred to as “EK100_5X”), and 10X ergostatrien-3β-ol group (referred to as “EK100_10X”) are shown.

FIG. 12 is a bar diagram showing the effects of alcohol consumption and ergostatrien-3β-ol intake on the expressions of genes of alcohol metabolism-related enzymes (i.e., ADH, ALDH, CYP2E1, and CAT) in the livers of the mice, wherein the results of the control group (referred to as “control”), alcohol-treated group (referred to as “EtOH”), 1X ergostatrien-3β-ol group (referred to as “EK100_1X”), 5X ergostatrien-3β-ol group (referred to as “EK100_5X”), and 10X ergostatrien-3β-ol group (referred to as “EK100_10X”) are shown.

FIGS. 13A and 13B show the effects of alcohol consumption and ergostatrien-3β-ol intake on the expression of CYP2E1 protein in the livers of the mice, wherein FIG. 13A is a photograph showing the results of western blotting, and FIG. 13B is a bar diagram showing the quantitative results. FIGS. 13A and 13B both include the results of the control group (referred to as “control”), alcohol-treated group (referred to as “EtOH”), 1X ergostatrien-3β-ol group (referred to as “EK100_1X”), 5X ergostatrien-3β-ol group (referred to as “EK100_5X”), and 10X ergostatrien-3β-ol group (referred to as “EK100_10X”).

FIGS. 14A and 14B show the effects of alcohol consumption and ergostatrien-3β-ol intake on the activities of ADH and ALDH in the livers of the mice, wherein FIG. 14A is a bar diagram showing the activities of ADH, and FIG. 14B is a bar diagram showing the activities of ALDH. FIGS. 14A and 14B both include the results of the control group (referred to as “control”), alcohol-treated group (referred to as “EtOH”), 1X ergostatrien-3β-ol group (referred to as “EK100_1X”), 5X ergostatrien-3β-ol group (referred to as “EK100_5X”), and 10X ergostatrien-3β-ol group (referred to as “EK100_10X”).

FIG. 15 is a bar diagram showing the effects of alcohol consumption and ergostatrien-3β-ol intake on the concentrations of alcohol in the serum of the mice, wherein the results of the control group (referred to as “control”), alcohol-treated group (referred to as “EtOH”), 1X ergostatrien-3β-ol group (referred to as “EK100_1X”), 5X ergostatrien-3β-ol group (referred to as “EK100_5X”), and 10X ergostatrien-3β-ol group (referred to as “EK100_10X”) are shown.

### DESCRIPTION OF THE PREFERRED EMBODIMENTS

The following will describe some embodiments of the present invention in detail. However, without departing from the spirit of this invention, the present invention may be embodied in various embodiments and should not be illustrated as limited to the embodiments descried in the specification. In addition, unless otherwise indicated herein, the expressions “a,” “an,” “the,” or the like recited in this specification (especially in the claims) are intended to include both the singular and plural forms. The term “effective amount” or “therapeutically effective amount” used in this specification refers to the amount of compound that can at least partially alleviate the condition that is being treated in a suspected subject when administrated to the subject in need. The term “subject” used in this specification refers to a mammalian, including human or non-human animals.

Unless otherwise indicated herein, the term “ergostatrien-3β-ol” in this specification includes ergostatrien-3β-ol, a pharmaceutically acceptable ester of ergostatrien-3β-ol, and combinations thereof.

According to the classification of World Health Organization (WHO), excessive alcohol consumption refers to a man consuming over 8 units (8 g for each unit) of alcohol per week, or a woman consuming over 6 units of alcohol per week. If the alcohol consumption is over 21 units per week for a man or over 14 units per week for a woman, this is classified as hazardous drinking. In the present invention, the term “chronic alcohol consumption” refers to the continuous consumption of alcohol over a period of time (such as several weeks, several months, or even several years), wherein the amount of alcohol consumption would not be less than excessive alcohol consumption, but less than hazardous drinking.

Research has shown that long-term excessive alcohol consumption increases body fat and causes hepatic injuries, wherein the hepatic injuries include hepatitis, liver fibrosis, liver cirrhosis, or even hepatic carcinoma. It has also been shown that, if the hepatic alcohol metabolism capability increases, the hepatic lipid homeostasis modulates, the hepatic antioxidant capability increases, the production of hepatic oxidative free radicals decreases, and/or the hepatic anti-inflammatory effect increases, then the liver can be effectively protected, thereby alleviating, inhibiting and/or treating hepatic injuries caused by alcohol consumption. Relevant description can be seen in articles, such as “Some novel insights into the pathogenesis of alcoholic steatosis. Alcohol. 34: 45-48 (2004);” “Molecular mechanisms of alcoholic fatty liver. Alcohol Clin Exp Res. 33: 191-205 (2009);” “Alcohol and lipid metabolism. Am J Physiol Endocrinol Metab. 295: 10-16 (2008);” “The role of lipid metabolism in the pathogenesis of alcoholic and nonalcoholic hepatic steatosis. Seminar in Liver Disease. 30: 378-390 (2010);” “Chronic ethanol and nicotine interaction on rat tissue antioxidant defense system. Alcohol. 25: 89-97 (2001);” “Tumor necrosis factor in alcohol enhanced endotoxin liver injury. Alcohol Clin Exp Res. 16: 665-669 (1992);” “Alcohol and oxidative liver injury. Hepatology. 43: 63-74; Alcohol: its metabolism and interaction with nutrients. Annu Rev Nutr. 20: 395-430 (2000);” “Peroxisome proliferator-activated receptor α (PPARα) agonist treatment reverses PPARα dysfunction and abnormalities in hepatic lipid metabolism in ethanol-fed mice. J Biol Chem. 278: 27997-28004 (2003);” and “Protective effect of cyclooxygenase-2 (COX-2) inhibitors but not non-selective cyclooxygenase (COX)-inhibitors on ethanol withdrawal-induced behavioural changes. Addic Biol. 10: 329-335 (2005),” which are incorporated herein by reference.

In addition, if the lipogenesis in the body can be decreased or the body fatty acid metabolism can be increased, it will be favorable for decreasing body fat accumulation, and thus, alleviating and/or inhibiting the occurrence of various fatty acid metabolism-related diseases (such as hyperlipidemia, arteriosclerotic cardiovascular diseases, cardiac arrhythmia, heart failure, vascular obstruction, etc.).

The inventors of the present invention found that ergostatrien-3β-ol could provide the effects of modulating hepatic lipid homeostasis, increasing hepatic antioxidant capability, decreasing production of hepatic oxidative free radicals, increasing hepatic anti-inflammatory capability, and alleviating the condition of hepatic injuries in a subject with hepatic injuries caused by alcohol consumption.

Therefore, the present invention relates to the use of ergostatrien-3β-ol in the manufacture of a medicament, wherein the medicament is used for alleviating, inhibiting and/or treating hepatic injuries caused by alcohol consumption, especially for alleviating, inhibiting and/or treating hepatic injuries caused by chronic alcohol consumption. Examples of hepatic injuries include, but are not limited to, hepatitis, liver fibrosis, liver cirrhosis, and hepatic carcinoma. In an embodiment of the present invention, the medicament is used for alleviating, inhibiting and/or treating hepatitis caused by alcohol consumption.

The inventors of the present invention further found that ergostatrien-3β-ol could provide efficacy in alleviating body fat accumulation in a subject with body fat accumulation caused by alcohol consumption. Therefore, the present invention also relates to the use of ergostatrien-3β-ol in the manufacture of a medicament, wherein the medicament is used for alleviating and/or inhibiting body fat accumulation caused by alcohol consumption, especially for alleviating and/or inhibiting body fat accumulation caused by chronic alcohol consumption. In an embodiment of the present invention, the medicament is used for alleviating and/or inhibiting the hepatic fat accumulation (i.e., fatty liver).

It has been known that ergostatrien-3β-ol can be purified and isolated from Chinese herbal medicines (such as Antrodia camphorata), or can be obtained by chemical synthesis. For example, commercially available powder of Antrodia camphorata can be extracted using methanol, and separated by silica gel column chromatography with ethyl acetate/n-hexane (ethyl acetate:n-hexane=1:9) to eluent ergostatrien-3β-ol.

The medicament according to the present invention may be prepared in any suitable form depending on the desired administration manner. For example, the medicament can be administered by oral or parenteral (such as subcutaneous, intravenous, intramuscular, peritoneal, or nasal) route to a subject in need, but is not limited thereby. Depending on the form and purpose, a suitable carrier can be chosen and used to provide the medicament.

As a form suitable for oral administration, the medicament provided by the present invention may comprise any pharmaceutically acceptable carrier that will not adversely affect the desired effects of ergostatrien-3β-ol. Examples of suitable carriers can include, for example, solvents (water, saline, dextrose, glycerol, ethanol or its analogs, or combinations thereof), oily solvents, diluents, stabilizers, absorbent retarders, disintegrants, emulsifiers, antioxidants, adhesives, binders, tackifiers, dispersants, suspending agents, lubricants, hygroscopic agents, solid carriers (e.g., starch, bentonite), etc. The medicament can be provided in any suitable form for oral administration, such as in the form of a tablet (e.g., dragee), a pill, a capsule, a granule, a pelvis, a fluidextract, a solution, syrup, a suspension, an emulsion, and a tincture, etc.

As for the form of injection or drip suitable for subcutaneous, intravenous, intramuscular, or peritoneal administration, the medicament provided by the present invention may comprise one or more ingredient(s), such as an isotonic solution, a salt-buffered saline (e.g., phosphate-buffered saline or citrate-buffered saline), a hydrotropic agent, an emulsifier, 5% sugar solution, and other carriers to provide the medicament as an intravenous infusion, an emulsified intravenous infusion, a powder for injection, a suspension for injection, or a powder suspension for injection, etc. Alternatively, the medicament may be prepared as a pre-injection solid. The pre-injection solid can be provided in a form which is soluble in other solutions or suspensions, or in an emulsifiable form. A desired injection is provided by dissolving the pre-injection solid in other solutions or suspensions or emulsifying it prior to being administered to a subject in need. In addition, examples of the form for external use which are suitable for nasal or transdermal administration include an emulsion, a cream, gel (e.g., an aquagel), paste (e.g., a dispersion paste and an ointment), a spray, or a solution (e.g., a lotion and a suspension).

Optionally, the medicament provided by the present invention may further comprise a suitable amount of additives, such as a flavoring agent, a toner, or a coloring agent for enhancing the palatability and the visual perception of the medicament, and/or a buffer, a conservative, a preservative, an antibacterial agent, or an antifungal agent for improving the stability and storability of the medicament. In addition, the medicament may optionally further comprise one or more other active ingredient(s), or be used in combination with a medicament comprising one or more other active ingredients, to further enhance the effects of the medicament or to increase the application flexibility and adaptability of the preparation thus provided, as long as the other active ingredients will not adversely affect the desired effects of ergostatrien-3β-ol.

Depending on the age, body weight, and health conditions of the subject to be administrated, the medicament provided by the present invention may be applied with various administration frequencies, such as once a day, multiple times a day, or once every few days, etc. For example, when the medicament is applied orally to a subject for alleviating, inhibiting and/or treating hepatic injuries (e.g., hepatitis) caused by alcohol consumption, or when the medicament is applied orally to a subject for alleviating and/or inhibiting body fat accumulation (e.g., liver fat accumulation) caused by alcohol consumption, the dosage of the medicament is about 1 mg (as ergostatrien-3β-ol)/kg-body weight to about 100 mg (as ergostatrien-3β-ol)/kg-body weight per day, preferably about 5 mg (as ergostatrien-3β-ol)/kg-body weight to about 70 mg (as ergostatrien-3β-ol)/kg-body weight per day, and more preferably about 10 mg (as ergostatrien-3β-ol)/kg-body weight to about 40 mg (as ergostatrien-3β-ol)/kg-body weight per day, wherein the unit “mg/kg-body weight” refers to the dosage required per kg-body weight of the subject. However, for acute patients, the dosage may be optionally increased up to several folds or dozen folds, depending on the practical requirements.

Furthermore, the medicament of the present invention may be manufactured into a form (e.g., a tablet or a capsule) of “extended-release (also called sustained-release, SR),” “sustained-action (SA),” “time-release (TR),” “controlled-release (CR),” “modified release (MR),” or “continuous-release (CR)” to slowly dissolve with time and release the active ingredients comprised therein (i.e., ergostatrien-3β-ol and/or a pharmaceutically acceptable ester thereof), to decease the peak value of active ingredients in blood and maintain a stable level of active ingredients in blood for a long time with a lower use frequency.

The present invention further provides a method of alleviating, inhibiting and/or treating hepatic injuries caused by alcohol consumption, comprising administering to a subject in need an effective amount of ergostatrien-3β-ol, a pharmaceutically acceptable ester thereof, or combinations thereof. Especially, the present invention provides a method of alleviating, inhibiting and/or treating hepatic injuries caused by chronic alcohol consumption.

The present invention yet provides a method of alleviating and/or inhibiting body fat accumulation caused by alcohol consumption, comprising administering to a subject in need an effective amount of ergostatrien-3β-ol, a pharmaceutically acceptable ester thereof, or combinations thereof. Especially, the present invention provides a method of alleviating and/or inhibiting body fat accumulation caused by chronic alcohol consumption.

In the above two methods, the applied form, suitable dosage, applied subject, and applied manner of ergostatrien-3β-ol are all in line with the above description about the medicament of the present invention.

It has been found that ergostatrien-3β-ol can also increase the alcohol metabolism capability of the liver, and alleviate or prevent the disadvantages or inconveniences caused by alcohol consumption. Therefore, the present invention also relates to a use of ergostatrien-3β-ol and/or a pharmaceutically acceptable ester thereof in the manufacture of a preparation, wherein the preparation is used for increasing the alcohol metabolism capability of the liver.

The preparation provided according to the present invention may be a food additive for adding into foods, or be a food, and may be prepared in any suitable form (e.g., a solid or a fluid) without specific limitations. For example, if the preparation is provided as a food additive, the food additive may be in various forms such as a powder, a liquid, a suspension, or a granule, to be added conveniently during the manufacturing processes of foods; and if the preparation is provided as a food, the food may be, for example, dairy products, processed meat, breadstuff, pasta products, cookies, troches, fruit juices, teas, sport drinks, and nutritious drinks, or may be general daily food or health foods taken as dietary supplements or nutritious supplements after surgery, but is not limited thereby. The food can be eaten prior to, simultaneously with, and/or after the consumption of alcohol-containing substances, to increase the alcohol metabolism capability of the liver, and alleviate or prevent the disadvantages or inconveniences caused by alcohol consumption.

Depending on the age, body weight and healthy conditions of the subject to be administrated, the health food provided by the present invention may be taken in various frequencies, such as once a day, several times a day, or once every few days, etc. The amount of ergostatrien-3β-ol in the health food provided by the present invention may also be adjusted, preferably to the amount that should be taken daily, depending on the specific population. For example, if the recommended daily dosage for a subject is about 10 mg and each serving of the health food contains 5 mg of ergostatrien-3β-ol, the subject may take about two servings of the health food per day.

The recommended daily dosages, use standards and use conditions for a specific population (e.g., patients with hepatitis), or recommendations for a use in combination with another food or medicament can be labeled on the outer packaging of the health food of the present invention, and thus, is favorable for users to take the health food by him- or herself safely and securely without the directions of a doctor, pharmacist, or related executive.

To alleviate or prevent the disadvantages or inconveniences caused by alcohol consumption, the present invention also provides a method of increasing the alcohol metabolism capability of the liver, comprising administering to a subject in need an effective amount of ergostatrien-3β-ol, a pharmaceutically acceptable ester thereof, or combinations thereof. In this method, the applied form, suitable dosage, applied subject, and applied manner of ergostatrien-3β-ol are all in line with the above description about the preparation of the present invention.

The present invention will be further illustrated in detail with specific examples as follows. However, the following examples are provided only for illustrating the present invention, and the scope of the present invention is not limited thereby. The scope of the present invention will be indicated in the appended claims.

### EXAMPLES

### Preparation Examples

A. Preparation of Ergostatrien-3β-Ol

1.6 kg of freeze-dried Taiwanofungus camaphoratus mycelium powder (purchased from Grape King Bio) was extracted three times with methanol under room temperature, and the methanol extracts obtained therefrom were combined. The methanol extract was filtered by Whatman filter paper and then processed by vacuum concentration, to obtain a crude extract. After that, the crude extract was partitioned by liquid-liquid partition with ethyl acetate and water (wherein the volume ratio of ethyl acetate to water was 1:1), followed by removing the water phase and retaining the ethyl acetate phase. Thereafter, the remaining ethyl acetate phase was separated by column chromatography (stationary phase: SiO₂; eluent: ethyl acetate/n-hexane (1:9)), and the eluent was collected to obtain a white crystal and to conduct a recrystallization with acetone to obtain 5342.2 mg of ergostatrien-3β-ol with purity greater than 99%.

B. Preparation of Mice Model for Animal Experiment

40 male C57BL/6 mice (each of them was 8 weeks old and weighed about 20 to 22 g) were bred under an environment with a temperature of 22±2° C., a humidity of 60 to 80%, and a day-night circle of 12 hours for a week, and then separated randomly into 5 groups (each group had 8 mice). Then, the 5 groups of mice were bred under the following conditions, respectively, to conduct the experiment:
(1) Control group (referred to as “control”): ad libitum feeding with a normal liquid diet (the composition thereof is shown in Table 1) and gavage feeding with 0.1 mg of saline every day;
(2) Alcohol-treated group (referred to as “EtOH”): ad libitum feeding with a Lieber-DeCarli alcoholic liquid diet (the composition thereof is shown in Table 1) and gavage feeding with 0.1 mg of saline every day;
(3) 1X ergostatrien-3β-ol group (referred to as “EK100_1X”): ad libitum feeding with a Lieber-DeCarli alcoholic liquid diet and gavage feeding with ergostatrien-3β-ol (dosage: 1 mg/kg-body weight, dissolved in 0.1 ml of double deionized water) every day;
(4) 5X ergostatrien-3β-ol group (referred to as “EK100_5X”): ad libitum feeding with a Lieber-DeCarli alcoholic liquid diet and gavage feeding with ergostatrien-3β-ol (dosage: 5 mg/kg-body weight, dissolved in 0.1 ml of double deionized water) every day;
(5) 10X ergostatrien-3β-ol group (referred to as “EK100_10X”): ad libitum feeding with a Lieber-DeCarli alcoholic liquid diet, and gavage feeding with ergostatrien-3β-ol (dosage: 10 mg/kg-body weight, dissolved in 0.1 ml of double deionized water) every day.

TABLE 1

| | | Lieber-DeCarli |
|---|---|---|
| | Normal liquid diet | alcoholic liquid diet |
| Ingredients | Amount per liter | Amount per liter |
| Casein | 41.4 g | 41.4 g |
| L-cystine | 0.5 g | 0.5 g |
| DL-methionine | 0.3 g | 0.3 g |
| Corn oil | 8.5 g | 8.5 g |
| Olive oil | 28.4 g | 28.4 g |
| Safflower oil | 2.7 g | 2.7 g |
| Maltodextrin | 115.2 g | 25.6 g |
| Cellulose | 10 g | 10 g |
| Mixed salting agent #210011 | 8.75 g | 8.75 g |
| Mixed vitamins #310011 | 2.5 g | 2.5 g |
| Choline bitartrate | 0.53 g | 0.53 g |
| Xanthan gum | 3 g | 3 g |
| 95% alcohol | 0 | 67.3 mg |
| Water | Balance | Balance |

Note:
the above feed formulations can be seen in articles, such as “The Feeding of Alcohol in Liquid Diets: Two Decades of Applications and 1982 Update. Alcoholism-Clinical and Experimental research. 6: 523-531 (1982).” Each diet may provide 1,000 calories per milliliter.

C. Observation, Recording, and Sample Collection of Mice Model

C-1. The feed intakes of mice were recorded every day (4 mice from each group were randomly picked for recording, and an average of the 4 mice was taken). The changes in the mice body weight were recorded at the last day of every week (i.e., an average of 8 mice from each group), over a four-week period.

C-2. At the end of week 3, the feces of mice were collected (for all of the 8 mice from each group) and then dried by an oven to store for the following analysis.

C-3. At the end of week 4, the mice were fasted for 8 hours. Then, mice blood was collected by orbital sinus blood collection with capillary blood collection needles (for all of the 8 mice from each group). The mice blood was left standing for 1 hour and then centrifuged by a refrigerated microcentrifuge (Kubota corporation, model number: 3700) to separate the supernatant serum. The supernatant serum was stored at −80° C. for the following analysis.

C-4. After “C-3” was completed, the mice were sacrificed. The weights of the mice hearts, livers, kidneys, spleens, and abdominal and epididymal fat pads were measured and recorded; the mice livers were washed with sterilized saline, and then the liver appearances were recorded by a camera. The collected organs and feces were stored at −80° C. for the following analysis (recording, sample collection and storage were conducted for all of the 8 mice from each group).

D. Sample Processing and Analysis

D-1. Preparation of Lipid Extracts of Mouse Liver or Feces

The feces samples of each group provided by “C-3” or the liver samples of each group provided by “C-4” were placed in glass test tubes and processed as follows, respectively: (i) an extraction solution (prepared by methane and methanol with a volume ratio of 2:1) was added into the tube; (ii) the mixture obtained therefrom was ultrasonically oscillated and then filtered with filter paper; (iii) the light-yellow solution obtained therefrom was put into another glass test tube to repeat the above extraction steps until the obtained liquid was colorless; (iv) then, the colorless liquid was dried with the use of nitrogen, and the yellow substrate obtained therefrom was lipid; (v) isopropanol was added and the mixture obtained therefrom was ultrasonically oscillated to resolve the lipid completely and obtain a lipid extract for the following analysis.

D-2. Extraction and Analysis of mRNA from Mouse Liver

0.1 g of mouse liver sample of each group provided by [Preparation Examples] C-4 was put into a 0.8 ml RNAlater containing an RNA stabilizer (Qiagen company, United States), and then the commercial kit (E.Z.N.A™ Tissue RNA kit, purchased from Omega Bio-Tek company, United States) was used to extract the mRNA therein. After that, the mRNA sample obtained therefrom was reverse transcribed to provide cDNA, and then specific primers (as shown in Table 2) were used to conduct real-time polymerase chain reaction (real-time PCR) of the cDNA to observe the expression of each gene in the mouse liver, wherein the operation steps of the real-time polymerase chain reaction were as follows: (i) 2 μl of cDNA (50 ng/μl), 5 μl of SYBR Green (Molecular Probes company, United States, Applied Biosystems/Fast SYBR® Green Master Mix), 2 μl of RNase-free water, 1 μl of forward primer, and 1 μl of reverse primer were well mixed and then centrifuged; (ii) the above sample was put into a real-time polymerase chain reaction system (Applied Biosystems/StepOne Real time PCR system) to detect the expression of each gene, wherein the expression of GAPDH was used as the positive control.

TABLE 2

| | | Sequence |
|---|---|---|
| Name of gene | Nucleotide sequence of primer | number |
| GAPDH | (Forward primer) AACCTGCCAAGTATGATGA | SEQ ID NO: 1 |
| (XM_003945995.1) | (Reverse primer) GGAGTTGCTGTTGAAGTC | SEQ ID NO: 2 |
| LXR-α | (Forward primer) GCTCTGCTCATAGCCATCAG | SEQ ID NO: 3 |
| (NM_031627.2) | (Reverse primer) CAGGGCCTCCACATATGTGT | SEQ ID NO: 4 |
| SREBP-1c | (Forward primer) CACAGCGGTTTTGAACGACA | SEQ ID NO: 5 |
| (NM_011480.3) | (Reverse primer) CTCTCAGGAGAGTTGGCACC | SEQ ID NO: 6 |
| ACC | (Forward primer) GGAGGCTGCATTGAACACAAG | SEQ ID NO: 7 |
| (NM_133904.2) | (Reverse primer) CGACGGTGAAATCTCTGTGC | SEQ ID NO: 8 |
| FAS | (Forward primer) GCTGCGGAAACTTCAGGAAAT | SEQ ID NO: 9 |
| (NM_007988.3) | (Reverse primer) AGAGACGTGTCACTCCTGGACTT | SEQ ID NO: 10 |
| ME | (Forward primer) AACTCTGACTTCGACAGGTATCT | SEQ ID NO: 11 |
| (NM_001198933.1) | (Reverse primer) CGGAATGCCAAACTGTACTGC | SEQ ID NO: 12 |
| PPAR-α | (Forward primer) TGACACCTTCCTCTTCCCAAA | SEQ ID NO: 13 |
| (NM_001113418.1) | (Reverse primer) CGTCGGACTCGGTCTTCTTG | SEQ ID NO: 14 |
| RXR-α | (Forward primer) CCAAACATTTCCTGCCGCTC | SEQ ID NO: 15 |
| (NM_011305.3) | (Reverse primer) CGACCCGTTGGAGAGTTGAG | SEQ ID NO: 16 |
| CPT1 | (Forward primer) CTGAGCCATGAAGCCCTCAA | SEQ ID NO: 17 |
| (NM_013495.2) | (Reverse primer) CACACCCACCACCACGATAA | SEQ ID NO: 18 |
| UCP2 | (Forward primer) ACAAGACCATTGCACGAGAG | SEQ ID NO: 19 |
| (NM_011671.4) | (Reverse primer) ATGAGGTTGGCTTTCAGGAG | SEQ ID NO: 20 |
| TLR4 | (Forward primer) ACCAGGAAGCTTGAATCCCTG | SEQ ID NO: 21 |
| (NM_021297.2) | (Reverse primer) TCATCAGGGACTTTGCTGAGTT | SEQ ID NO: 22 |
| MyD88 | (Forward primer) CATGGTGGTGGTTGTTTCTGAC | SEQ ID NO: 23 |
| (NM_010851.2) | (Reverse primer) CTGGAGACAGGCTGAGTGCAA | SEQ ID NO: 24 |
| NF-κB | (Forward primer) CCGTGTTTGTTCAGCTTCGG | SEQ ID NO: 25 |
| (NM_008689.2) | (Reverse primer) CTGTCCGAGAAGTTCGGCAT | SEQ ID NO: 26 |
| iNOS | (Forward primer) GGCAGCCTGTGAGACCTTTG | SEQ ID NO: 27 |
| (NM_010927.3) | (Reverse primer) GCATTGGAAGTGAAGCGTTTC | SEQ ID NO: 28 |
| COX-2 | (Forward primer) TGGGTTCACCCGAGGACTG | SEQ ID NO: 29 |
| (NM_011198.3) | (Reverse primer)GGGGATACACCTCTCCACCAA | SEQ ID NO: 30 |
| α-SIVL4 | (Forward primer)TTCGTGACTACTGCCGAGCGTGAGA | SEQ ID NO: 31 |
| (NM_007392.2) | (Reverse primer) AAAGATGGCTGGAAGAG | SEQ ID NO: 32 |
| ADH | (Forward primer) GGCCGCCTTGACACCAT | SEQ ID NO: 33 |
| (NM_007409.2) | (Reverse primer) GCACTCCTACGACGACGCTTA | SEQ ID NO: 34 |
| ALDH | (Forward primer) CGAACGTCTGCCCTATCAACTT | SEQ ID NO: 35 |
| (NM_009656.3) | (Reverse primer) CCGGAATCGAACCCTGATT | SEQ ID NO: 36 |
| CYP2E1 | (Forward primer) GCCCGCATCCAAAGAGA | SEQ ID NO: 37 |
| (NM_021282.2) | (Reverse primer) GGCTGGCCTTTGGTCTTTT | SEQ ID NO: 38 |
| CAT | (Forward primer) TGAGAAGCCTAAGAACGCAATT | SEQ ID NO: 39 |
| (NM_009804.2) | (Reverse primer) CCCTTCGCAGCCATGTG | SEQ ID NO: 40 |

D-3. Preparation of the Mouse Liver Homogenate

0.3 g of mouse liver sample of each group provided by [Preparation Examples] C-4 was taken, and 2.7 ml of phosphate-buffered saline (PBS) was added therein. After homogenizing with a homogenizer (Polytron, Switzerland, PT-2100), it was centrifuged (4° C., 3000 rpm, 15 minutes). The supernatant was filtered with a filter paper (ADVANTEC, NO. 1 55 mm), and the filtrate obtained therefrom was 10% liver homogenate. After analyzing the concentrations of proteins with the Bio-rad protein assay, it was stored at −20° C. for the following analysis.

D-4. Staining of the Mouse Liver Samples

Hematoxylin-eosin (H&E) stain of the mouse liver samples of each group provided by [Preparation Examples] C-4 was conducted, wherein the operation steps were as follows: (i) a 1 cm³ tissue block was taken from each liver sample and immersed in 10% neutral buffered formalin; (ii) the sample obtained therefrom was dehydrated by alcohol with different concentrations sequentially in an order of 30%, 50%, 75%, 80%, 90%, 95%, and 100%; (iii) hyalinization of the sample was conducted by treating with xylene solution; (iv) the sample was embedded with paraffin wax solution; (v) the wax block obtained therefrom was sliced into sections with a thickness of about 5 μm by a slicer, the sections were put into warm water (about 40° C.), and then the expanded sections were dried, to provide the liver tissue sections; (vi) the above sections were dewaxed by treating with xylene solution for 20 minutes; (vii) the sections were immersed in alcohol with different concentrations sequentially in an order of 100%, 95%, 90%, 80%, 75%, 50%, 30%, for 15 minutes each; (viii) the sections were taken out and immersed in deionized water for 10 minutes to rehydrate the tissue; (ix) the rehydrated sections were placed in the hematoxylin stain for 30 seconds and then washed with deionized water; (x) the sections were immersed in the eosin stain for 2 to 5 minutes and then washed with deionized water; (xi) the sections were immersed in alcohol with different concentrations sequentially in an order of 30%, 50%, 75%, 80%, 90%, 95%, and 100% for dehydration; (xii) the sections were hyalinized by treating with xylene solution, and then mounted by Arabia gum, and observed under a microscope.

### Example 1: Observation of the Physiological Parameter Changes of Mice

To understand the physiological effects of alcohol consumption and ergostatrien-3β-ol intake on mice, the mice feed intake, change of body weight, weight of organ, and weight of fat were compared.

1-1. Change of Mice Body Weight

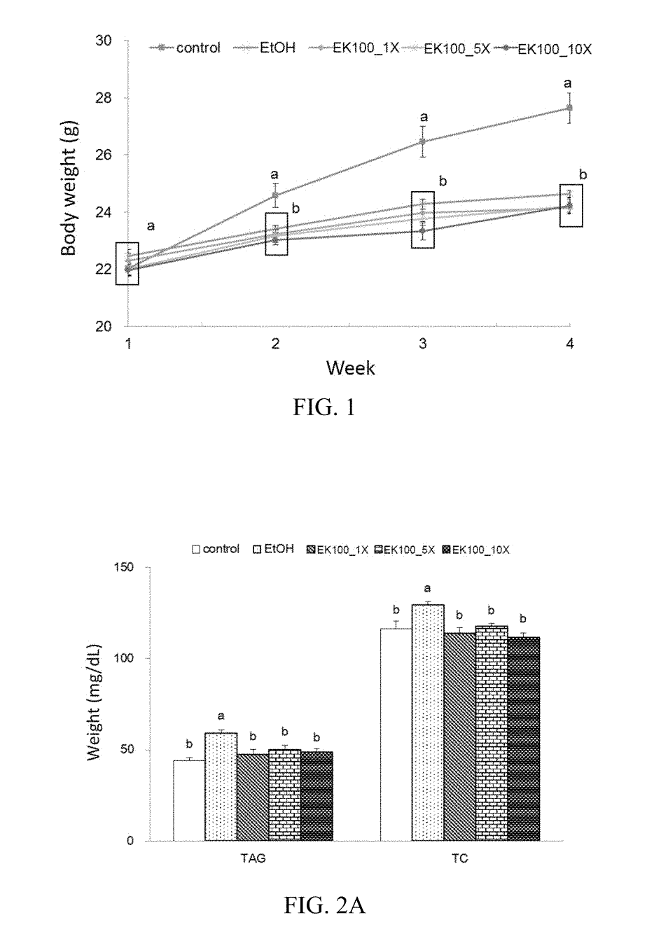

The body weights of mice of each group recorded every week in [Preparation Examples] C-1 were averaged, respectively, and the results are shown in FIG. 1. As shown in FIG. 1, in week 1 of the experiment, the body weights of mice from each group did not show any significant differences from each other (p>0.05). However, in week 2 and week 4 of the experiment, as compared to the “control,” the body weights of mice from the “EtOH,” “EK100_1X,” “EK100_5X,” and “EK100_10X” were significantly lower (p<0.05).

The above results indicate that alcohol consumption can cause a decrease in body weight, and the reasons thereof may be that, as compared to energy from non-alcoholic resources, energy from alcohol is more difficult to store, such that the energy from alcohol could enter the metabolic pathway preferentially and block other metabolic pathways at the same time. Although alcohol can provide high calories (about 7 kcal/g), it does not provide any nutrients. Therefore, high alcohol consumption may induce nutritional disorders and cause decreased body weight. Besides, research has shown that alcohol would cause damage to mitochondrial functions, decrease ATP synthesis, and result in energy deficiency in the body. This is probably the reason for the decrease in body weight caused by alcohol consumption. Relevant description can be seen in articles such as “The inhibition of gluconeogenesis following alcohol in humans. Am J Physiol. 275(5 Pt 1): 897-907 (1998);” “Acute effects of ethanol and acetate on glucose kinetics in normal subjects. Am J Physiol. 254(2 Pt 1): 175-180 (1998);” and “Alcoholic liver disease: pathology, pathogenetic and clinical aspects. Alcohol Clin Exp Res. 15(1): 45-66 (1991),” which are incorporated herein by reference.

1-2. Feed Intake of Mouse, Weight of Mouse Organ, and Weight of Mouse Fat

The average values of daily feed intakes of mice of each group recorded in [Preparation Examples] C-1 are shown in Table 3. Besides, the weights of mice organ and fat of each group obtained from [Preparation Examples] C-4 were averaged, respectively, and then the average weights (g) were divided by the average body weights of mice in week 4 from the corresponding group to obtain the relative weights of organs and the relative weights of fat. The results are also shown in Table 3.

TABLE 3

| | Control | EtOH | EK100_1X | EK100_5X | EK100_10X |
|---|---|---|---|---|---|
| Feed intake | 10.14 ± 0.07^{a} | 6.87 ± 0.06^{bc} | 6.57 ± 0.10^{c} | 7.09 ± 0.04^{b} | 6.72 ± 0.21^{c} |
| (g/each | | | | | |
| mouse/day) | | | | | |
| Relative weight | 3.38 ± 0.04^{b} | 4.55 ± 0.04^{a} | 4.50 ± 0.14^{a} | 4.33 ± 0.20^{a} | 4.48 ± 0.10^{a} |
| of liver | | | | | |
| (g/100 g of mice | | | | | |
| body weight) | | | | | |
| Relative weight | 0.49 ± 0.01^{c} | 0.64 ± 0.04^{a} | 0.53 ± 0.03^{bc} | 0.57 ± 0.04^{ab} | 0.51 ± 0.01^{bc} |
| of heart | | | | | |
| (g/100 g of mice | | | | | |
| body weight) | | | | | |
| Relative weight | 1.03 ± 0.03^{c} | 1.37 ± 0.03^{a} | 1.33 ± 0.04^{a} | 1.28 ± 0.05^{a} | 1.14 ± 0.02^{b} |
| of kidney | | | | | |
| (g/100 g of mice | | | | | |
| body weight) | | | | | |
| Relative weight | 0.38 ± 0.02^{b} | 0.62 ± 0.05^{a} | 0.53 ± 0.03^{a} | 0.55 ± 0.04^{a} | 0.55 ± 0.02^{a} |
| of spleen | | | | | |
| (g/100 g of mice | | | | | |
| body weight) | | | | | |
| Relative weight | 0.66 ± 0.06^{a} | 0.56 ± 0.06^{a} | 0.21 ± 0.03^{c} | 0.41 ± 0.06^{b} | 0.40 ± 0.04^{b} |
| of abdominal fat | | | | | |
| pad (g/100 g of | | | | | |
| mice body weight) | | | | | |
| Relative weight | 2.81 ± 0.15^{a} | 2.35 ± 0.14^{b} | 1.30 ± 0.11^{c} | 1.42 ± 0.14^{c} | 1.62 ± 0.07^{c} |
| of epididymal fat | | | | | |
| pad (g/100 g of | | | | | |
| mice body weight) | | | | | |

Note: in each column, if the results of different groups are significantly different from each other (P value smaller than 0.05), those results are represented by different letters. For example, the relative weight of liver of the “control” was 3.38±0.04^{b} (g/100 g of mice body weight) and the relative weight of liver of the “EtOH” was 4.55±0.04^{a} (g/100 g of mice body weight), indicating that the relative weights of liver of these two groups are significantly different (P value smaller than 0.05).

Table 3 shows that, as compared to the “control,” the feed intakes of mice from the “EtOH,” “EK100_1X,” “EK100_5X,” and “EK100_10X” were significantly lower (p<0.05). As for the relative weights of organs (e.g., liver, heart, kidney, and spleen), as compared to the “control,” the weights of mice liver, heart, kidney, and spleen were all significantly increased (p<0.05).

As for the relative weights of fat, Table 3 shows that there were no significant differences between the relative weights of abdominal fat pad in the “control” and the “EtOH”; however, as compared to the “EtOH,” the relative weights of epididymal fat pad of mice from the “EK100_1X,” “EK100_5X,” and “EK100_10X” were significantly lower (p<0.05).

The above results indicate that the feed intake of the “EtOH” mice was lower than that of the “control” mice, but the relative weights of liver, heart, kidney, and spleen of the “EtOH” mice were higher than those of the “control” mice, and the amounts of fat accumulated in the abdomen of the “EtOH” mice were the same of the “control” mice. The aforementioned phenomena may be due to the organ fat accumulation caused by alcohol consumption, such that the relative weights of organs (e.g., liver, heart, kidney, and spleen) for the “EtOH” mice were increased. The above results also indicate that ergostatrien-3β-ol is effective in alleviating or inhibiting body fat accumulation caused by alcohol consumption, and has effects on modulating the body lipid homeostasis.

### Example 2: Analysis of the Lipid Homeostasis in Mice Body

To understand the effects of alcohol consumption and ergostatrien-3β-ol intake on the lipid homeostasis in mice body, comparisons were made of the amounts of triacylglycerol (TAG) and total cholesterol (TC) in the mice serum and liver; the excreted amounts of TAG, TC and bile acid in the mice feces; and the expressions of lipid synthesis-related genes and fatty acid β-oxidation-promoting genes in mice liver. The appearance and the staining results of mice liver were also observed.

2-1. Amounts of TAG and TC in Serum

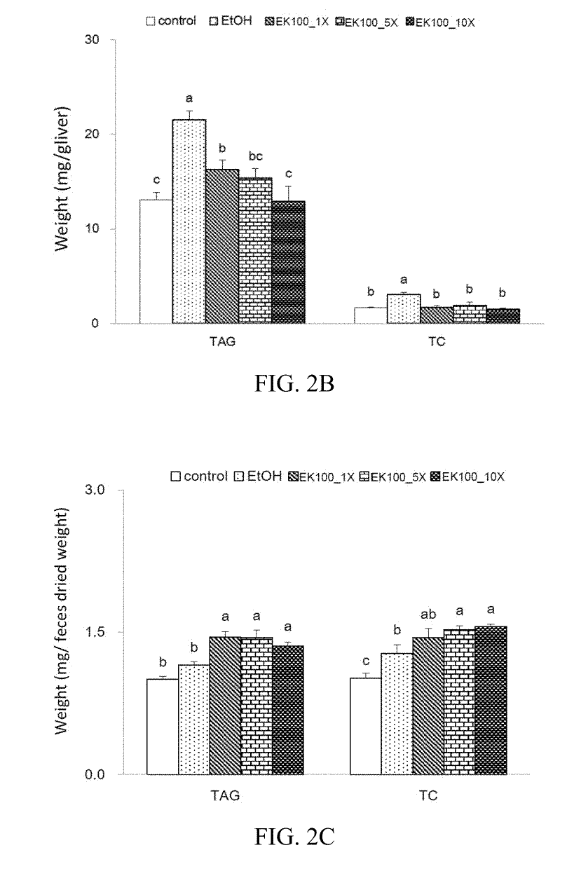

The amounts of TAG and TC in mice serum of each group provided by [Preparation Examples] C-2 were analyzed by a commercial kit (kit modal number: TR210, purchased from Randox Laboratories company of England). The results are shown in FIG. 2A, wherein, if the results of different groups are significantly different from each other (P value is smaller than 0.05), those results are represented by different letters. For example, “a” and “b” are shown in the results of the TAG amount in serum of the “control” mice and the “EtOH” mice respectively, indicating that the TAG amounts in the serum of these two groups are significantly different (P value smaller than 0.05). As shown in FIG. 2A, as compared to the “control” mice, the amounts of TAG and TC in the serum of the “EtOH” mice were all significantly increased (p<0.05); however, as compared to “EtOH” mice, the amounts of TAG and TC in the serum of “EK100_1X,” “EK100_5X,” and “EK100_10X” mice were all significantly decreased (p<0.05), or even decreased to that of the “control” mice. The above results indicate that alcohol consumption can cause fat (i.e., TAG and TC) accumulation in serum, while such a phenomenon is effectively alleviated and/or inhibited by ergostatrien-3β-ol, and thus, ergostatrien-3β-ol is effective in alleviating and/or inhibiting body fat accumulation caused by alcohol consumption.

2-2. Amounts of TAG and TC in Liver

The amounts of TAG and TC in mouse liver lipid extract of each group provided by [Preparation Examples] D-1 were analyzed by a commercial kit (kit modal number: TR210, purchased from Randox Laboratories company of England). The results are shown in FIG. 2B, wherein, if the results of different groups are significantly different from each other (P value smaller than 0.05), those results are represented by different letters. As shown in FIG. 2B, as compared to the “control” mice, the amounts of TAG and TC in the liver of the “EtOH” mice were all significantly increased (p<0.05); however, as compared to the “EtOH” mice, the amounts of TAG and TC in the liver of the “EK100_1X,” “EK100_5X,” and “EK100_10X” mice were all significantly decreased (p<0.05), or even decreased to that of the “control” mice. The above results indicate that alcohol consumption can cause fat (i.e., TAG and TC) accumulation in liver, while such a phenomenon is effectively alleviated or inhibited by ergostatrien-3β-ol. This indicates that ergostatrien-3β-ol is effective in alleviating and/or inhibiting liver fat accumulation caused by alcohol consumption.

2-3. Amounts of TAG, TC, and Bile Acid in Mouse Feces

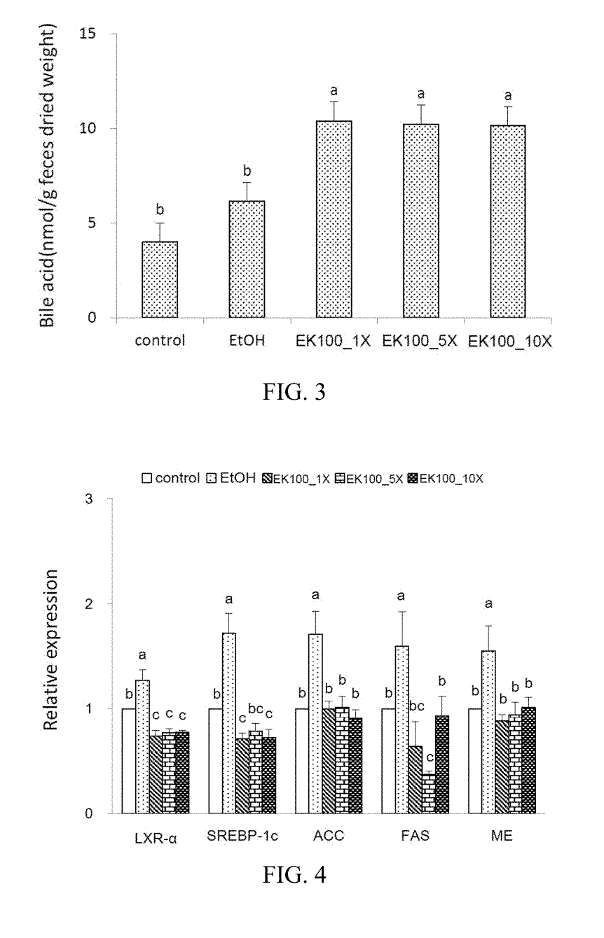

The amounts of TAG, TC, and bile acid in mouse liver lipid extract of each group provided by [Preparation Examples] D-1 were analyzed by a commercial kit (kit modal number: TR210, purchased from Randox Laboratories company of England). The results are shown in FIGS. 2C and 3, wherein, if the results of different groups are significantly different from each other (P value smaller than 0.05), those results are represented by different letters. As shown in FIGS. 2C and 3, as compared to the “EtOH” mice, the amounts of TAG, TC, and bile acid in the feces of the “EK100_1X,” “EK100_5X,” and “EK100_10X” mice were all significantly increased (p<0.05). The above results indicate that ergostatrien-3β-ol is effective in alleviating or inhibiting body fat accumulation (e.g., liver fat accumulation) caused by alcohol consumption by promoting the metabolism and excretion of lipid and cholesterol and decreasing the uptake of lipid and cholesterol.

2-4. Expressions of Lipid Synthesis-Related Genes in Liver

It has been known that LXR-α, SREBP-1c, ACC, FAS, ME, etc. are genes related to lipid synthesis, thus, the expressions of the above genes in mouse liver of each group were compared by the analysis method of [Preparation Examples] D-2. The results are shown in FIG. 4, wherein if the results of different groups are significantly different from each other (P value smaller than 0.05), those results are represented by different letters. As shown in FIG. 4, as compared to the “control” mice, the expressions of lipid synthesis-related genes in the liver of “EtOH” mice were all significantly increased (p<0.05); however, as compared to the “EtOH” mice, the expressions of lipid synthesis-related genes in the liver of the “EK100_1X,” “EK100_5X,” and “EK100_10X” mice were all significantly decreased (p<0.05), or even decreased to or lower than that of the “control” mice. The above results indicate that alcohol consumption can promote the expressions of lipid synthesis-related genes in liver and increase lipid synthesis, and thus, cause liver fat accumulation, while ergostatrien-3β-ol is effective in inhibiting the expressions of those genes and decreasing lipid synthesis, and thus, ergostatrien-3β-ol can effectively alleviate and/or inhibit liver fat accumulation caused by alcohol consumption, which contributes to the modulation of liver lipid homeostasis.

2-5. Expressions of Fatty Acid α-Oxidation-Promoting Genes in Liver

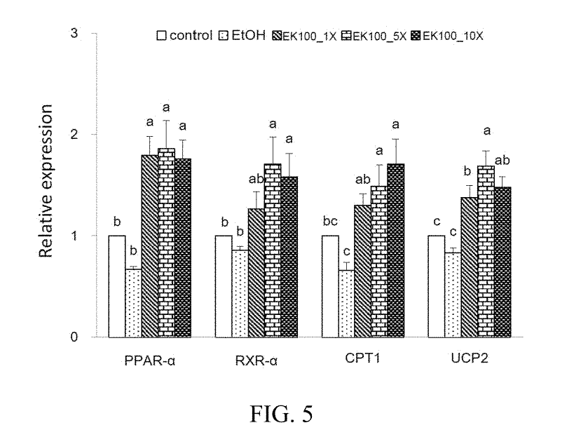

It has been known that fatty acid β-oxidation is closely related to the metabolism of fatty acids, and that PPAR-α, RXR-α, CPT1, and UCP2, etc. all belong to fatty acid β-oxidation-promoting genes. Therefore, the expressions of the above genes in the mouse liver of each group were compared by the analysis method of [Preparation Examples] D-2. The results are shown in FIG. 5, wherein, if the results of different groups are significantly different from each other (P value smaller than 0.05), those results are represented by different letters. As shown in FIG. 5, as compared to the “control” mice, the expressions of fatty acid β-oxidation-promoting genes in the liver of the “EtOH” mice all had a tendency to decrease; however, as compared to the “EtOH” mice, the expressions of fatty acid β-oxidation-promoting genes in the liver of the “EK100_1X,” “EK100_5X,” and “EK100_10X” mice were significantly increased (p<0.05). The above results indicate that alcohol consumption can decrease the expressions of fatty acid β-oxidation-promoting genes in the liver and decrease the metabolism of liver fatty acid, and thus, cause liver fat accumulation, while ergostatrien-3β-ol is effective in increasing the expressions of fatty acid β-oxidation-promoting genes and promoting the metabolism of liver fatty acids, and thus, ergostatrien-3β-ol can effectively alleviate and/or inhibit liver fat accumulation caused by alcohol consumption, which contributes to the modulation of liver lipid homeostasis.

2-6. Observation of the Appearance and Staining Results of Mouse Liver

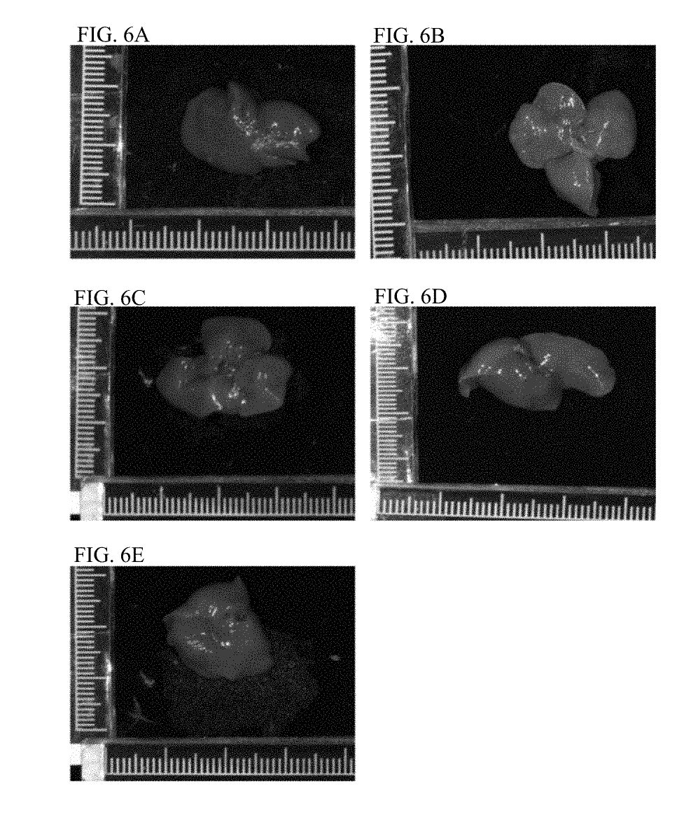

The photographs of the mouse liver appearance of each group recorded in [Preparation Examples] C-4 are shown in FIGS. 6A to 6E. As shown in FIGS. 6A to 6E, as compared to the “control” mice, the color of the liver of the “EtOH” mice was whiter (FIG. 6B), showing that the “EtOH” mice had already formed fatty liver; however, as compared to the “EtOH” mice, the whitening phenomenon of liver of the “EK100_1X,” “EK100_5X,” and “EK100_10X” mice decreased, indicating that ergostatrien-3β-ol is effective in alleviating, inhibiting and/or treating fatty liver caused by alcohol consumption.

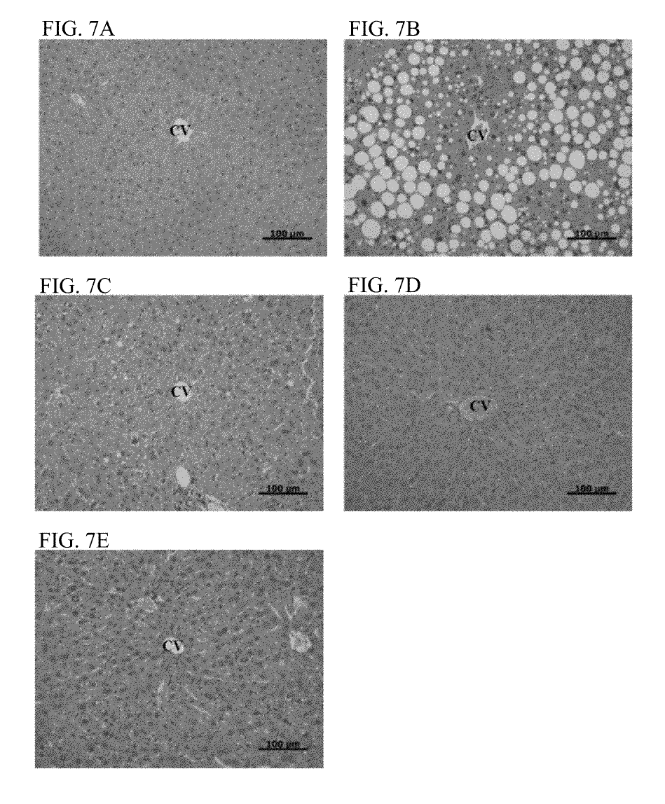

Additionally, the photographs of stained mouse liver of each group recorded in [Preparation Examples] C-4 are shown in FIGS. 7A to 7E, wherein the word “CV” refers to “central vein.” As shown in FIGS. 7A to 7E, as compared to the “control” mice, the number of fat vacuoles in the liver tissue of the “EtOH” mice was markedly increased, wherein fat vacuoles are a sign of fat accumulation in liver cells. However, as compared to the “EtOH” mice, the numbers of fat vacuoles in the liver tissue of the “EK100_1X,” “EK100_5X,” and “EK100_10X” mice were markedly decreased. The above results indicate again that alcohol consumption can promote liver fat accumulation, thereby causing fatty liver, while ergostatrien-3β-ol is effective in alleviating and/or inhibiting liver fat accumulation caused by alcohol consumption, and thus, ergostatrien-3β-ol can alleviate, inhibit and/or treat fatty liver caused by alcohol consumption.

### Example 3: Detection of Organ Injuries in the Mice

It has been known that both aspartate aminotransferase (AST) and alanine aminotransferase (ALT) are amino acid metabolic enzymes, which are widely present in the heart, liver, skeletal muscle, kidney and brain. When cells in organs are injured, AST and ALT will be released into the blood. Therefore, the activities of AST and ALT can be used as the injury markers of the above organs to detect organ injuries, wherein the higher activities of AST and ALT represent that organ injuries are more serious. For example, there is much more ALT in the liver. A higher activity of ALT in the liver would indicate a more serious injury.

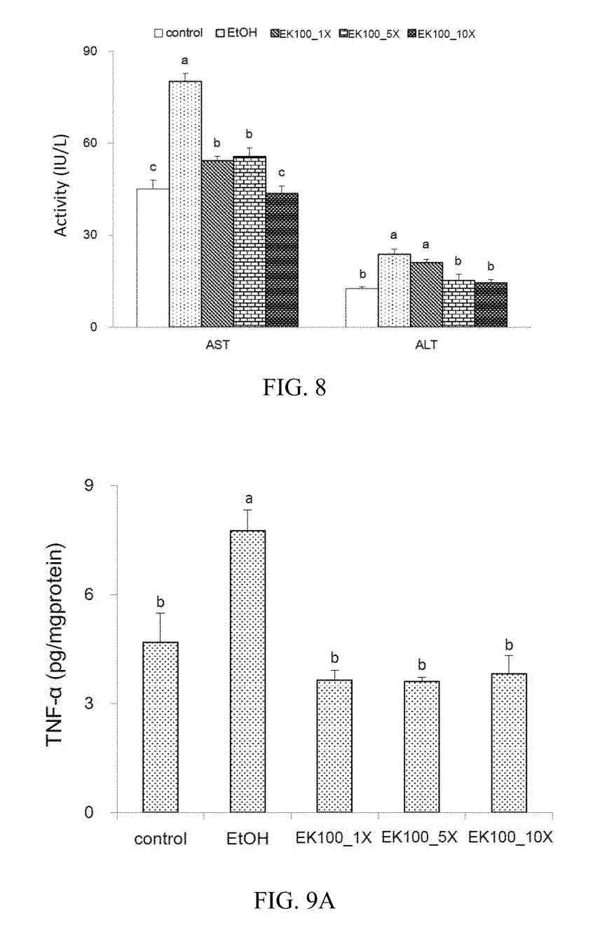

To understand the effects of alcohol consumption and ergostatrien-3β-ol intake on the injury conditions of mice organs, mouse serum samples of each group provided in [Preparation Examples] C-2 (200 μl per group) were placed into automatic dry-biochemistry analyzer (Arkray, Inc., Japan, Spotechem™ EZ, SP-4430) with AST/ALT expert analytic test paper (Arkray, Inc., Japan, Spotechem™ II) to detect the activities of AST and ALT. The results are shown in FIG. 8, wherein if the results of different groups are significantly different from each other (P value smaller than 0.05), those results are represented by different letters.

As shown in FIG. 8, as compared to the “control” mice, the activities of AST and ALT in the serum of the “EtOH” mice were significantly increased (p<0.05); however, as compared to the “EtOH” mice, the activities of AST in the serum of the “EK100_1X,” “EK100_5X,” and “EK100_10X” mice and the activities of ALT in the serum of the “EK100_5X,” and “EK100_10X” mice were significantly decreased (p<0.05), or even decreased to that of the “control” mice. The above results indicate that alcohol consumption can cause organ (especially liver) injuries, while ergostatrien-3β-ol is effective in alleviating the organ injuries caused by alcohol consumption.

### Example 4: Evaluation of the Oxidative Stress and the Anti-Oxidation Capability of Mouse Liver

It has been known that thiobarbituric acid reactive substances (TBARS), which produces malondialdehyde (MDA) after being oxidized, is a marker of lipid peroxidation. A higher TBARS value (i.e., amount of malondialdehyde) represents a higher level of lipid peroxidation. The abbreviation TEAC stands for trolox equivalent antioxidant capacity, wherein a higher TEAC value represents higher free radical-scavenging and anti-oxidant capabilities. Glutathione (GSH) is the first line of defense against the attack of free radicals, and if the amount of GSH decreases, the oxidative stress will increase. Superoxide dismutase (SOD), catalase (CAT), and glutathione peroxidase (GPx) all are important anti-oxidation enzymes, wherein the SOD converts the reactive oxygen species (ROS) into H₂O₂, which has a lower reactivity, and then the H₂O₂ is converted into oxygen and water by CAT and GPx.

To understand the effects of alcohol consumption and ergostatrien-3β-ol intake on mouse liver lipid peroxidation, oxidative stress, and anti-oxidation capability, the TBARS value, TEAC value, amount of GSH, activity of SOD, activity of CAT, and activity of GPx were compared between each group of mice.

4-1. TBARS Value

1 ml of mouse liver homogenates of each group provided in [Preparation Examples] D-3 were taken respectively, and 8.5 ml of trichloroacetic acid (TCA, purchased from J.T. Barker company, United States) was added therein respectively. After the extra protein had precipitated, 1.5 ml of TBA (2-thiobarbituric acid, purchased from Sigma CO company, United States) was added respectively and well mixed. After standing in a 95° C. water bath for 30 minutes, the mixture was centrifuged (4° C., 10000 rpm, 5 minutes). Then, the supernatant obtained therefrom was collected, and the absorbance thereof was analyzed by a spectrophotometer at 535 nm wavelength. The TBARS value (i.e., amount of malondialdehyde) was calculated through the formula 1 below. The results are shown in Table 4.

$$TBARS value = absorbance \times \frac{705.15}{concentration of liver protein} ( \frac{nmol malondialdehyde}{mg protein} ) \qquad Formula 1$$

TABLE 4

| | TBARS value (nmol malondialdehyde/mg protein) |
|---|---|
| Control | 0.55 ± 0.05^{b} |
| EtOH | 1.22 ± 0.08^{a} |
| EK100_1X | 0.56 ± 0.04^{b} |
| EK100_5X | 0.54 ± 0.05^{b} |
| EK100_10X | 0.46 ± 0.04^{b} |

As shown in Table 4, as compared to the “control” mice, the TBARS value of the liver of the “EtOH” mice was significantly increased (p<0.05); however, as compared to the “EtOH” mice, the TBARS values of the liver of the “EK100_1X,” “EK100_5X,” and “EK100_10X” mice were significantly decreased (p<0.05), or even decreased to that of the “control” mice. These results indicate that alcohol consumption can induce the peroxidation of liver lipid, while ergostatrien-3β-ol is effective in alleviating such a phenomenon.

4-2. TEAC Value

The TEAC values of mouse liver homogenates of each group provided in [Preparation Examples] D-3 were analyzed, wherein the analysis method can be seen in articles, such as “A critical appraisal of the use of the antioxidant capacity (TEAC) assay in defining optimal antioxidant structures. Food Chemistry. 80: 409-414 (2003),” which is incorporated herein by reference. The results are shown in Table 5.

TABLE 5

| | TEAC value (nmol/mg protein) |
|---|---|
| Control | 74.45 ± 1.20^{a} |
| EtOH | 60.80 ± 2.10^{b} |
| EK100_1X | 69.18 ± 2.77^{a} |
| EK100_5X | 75.23 ± 2.61^{a} |
| EK100_10X | 70.50 ± 2.25^{a} |

As shown in Table 5, as compared to the “control” mice, the TEAC value of the liver of the “EtOH” was significantly decreased (p<0.05); however, as compared to the “EtOH” mice, the TEAC values of the liver of the “EK100_1X,” “EK100_5X,” and “EK100_10X” mice were significantly increased (p<0.05), or even increased to that of the “control” mice. These results indicate that alcohol consumption can decrease the anti-oxidation capability of the liver, while ergostatrien-3β-ol is effective in alleviating such a phenomenon.

4-3. Amount of Glutathione (GSH)

The amounts of GSH in mouse liver homogenates of each group provided in [Preparation Examples] D-3 were analyzed, wherein the analysis method can be seen in articles such as “Du-Zhong (Eucommia ulmoides Oliv.) leaves inhibits CC14-induced hepatic damage in rats. Food Chem Toxicol. 44: 1424-1431 (2006),” which is incorporated herein by reference. The results are shown in Table 6.

TABLE 6

| | Amount of GSH (nmol/mg protein) |
|---|---|
| Control | 52.58 ± 2.91^{b} |
| EtOH | 32.37 ± 2.30^{c} |
| EK100_1X | 49.08 ± 4.78^{b} |
| EK100_5X | 71.14 ± 4.17^{a} |
| EK100_10X | 77.21 ± 2.95^{a} |

As shown in Table 6, as compared to the “control” mice, the amount of GSH in the liver of the “EtOH” mice was significantly decreased (p<0.05); however, as compared to the “EtOH” mice, the amount of GSH in the liver of the “EK100_1X,” “EK100_5X,” and “EK100_10X” mice were significantly increased (p<0.05), or even increased to that of or much higher than that of the “control” mice. These results indicate that alcohol consumption can increase the oxidative stress in liver, while ergostatrien-3β-ol is effective in alleviating such a phenomenon.

4-4. Activities of Anti-Oxidation Enzymes in Mouse Liver

The activities of SOD, CAT, and GPx in the mouse liver homogenates of each group provided in [Preparation Examples] D-3 were analyzed, wherein the analysis method can be seen in articles such as “Regulation of the insulin antagonistic protein tyrosine phosphatase 1B by dietary Se studied in growing rats. J Nutr Biochem. 20: 235-247 (2009);” “Effects of Artemisia capillaris ethyl acetate fraction on oxidative stress and antioxidant enzyme in high-fat diet induced obese mice. Chem Biol Interact. 179: 88-93 (2009);” and “Du-Zhong (Eucommia ulmoides Oliv.) leaves inhibits CC14-induced hepatic damage in rats. Food Chem Toxicol. 44: 1424-1431 (2006),” which are incorporated herein by reference. The results are shown in Table 7.

TABLE 7

| | Activity of SOD | Activity of CAT | Activity of GPx |
|---|---|---|---|
| | (munit/mg protein) | (unit/mg protein) | (unit/mg protein) |
| Control | 74.23 ± 3.43^{c} | 115.35 ± 2.44^{d} | 80.03 ± 1.74^{c} |
| EtOH | 78.89 ± 3.45^{bc} | 122.52 ± 3.52^{cd} | 107.35 ± 2.48^{b} |
| EK100_1X | 77.56 ± 3.33^{c} | 175.80 ± 6.89^{a} | 114.16 ± 2.40^{b} |
| EK100_5X | 90.40 ± 6.16^{b} | 145.43 ± 5.77^{b} | 110.74 ± 3.52^{b} |
| EK100_10X | 118.47 ± 4.64^{a} | 137.16 ± 6.79^{bc} | 131.89 ± 6.83^{a} |

As shown in Table 7, as compared to the “EtOH” mice, the activity of SOD in the liver of the “EK100_10X” mice was significantly increased (p<0.05). As for the activity of CAT, as compared to the “EtOH” mice, the activities of CAT in the liver of the “EK100_1X,” “EK100_5X,” and “EK100_10X” mice were all significantly increased (p<0.05). As for the activity of GPx, as compared to the “EtOH” mice, the activity of GPx in the liver of “EK100_10X” mice was significantly increased (p<0.05). The above results indicate that ergostatrien-3β-ol is effective in improving the anti-oxidation capability of the liver.

### Example 5: Observation of the Inflammation of Mouse Liver

To understand the effects of alcohol consumption and ergostatrien-3β-ol intake on the inflammation of mouse liver, the amounts of the inflammatory cytokines and the expressions of the inflammation-related genes in the mouse liver, and hepatitis histological activity indices of mouse liver were compared between each group.

5-1. Amount of Pro-Inflammatory Cytokine

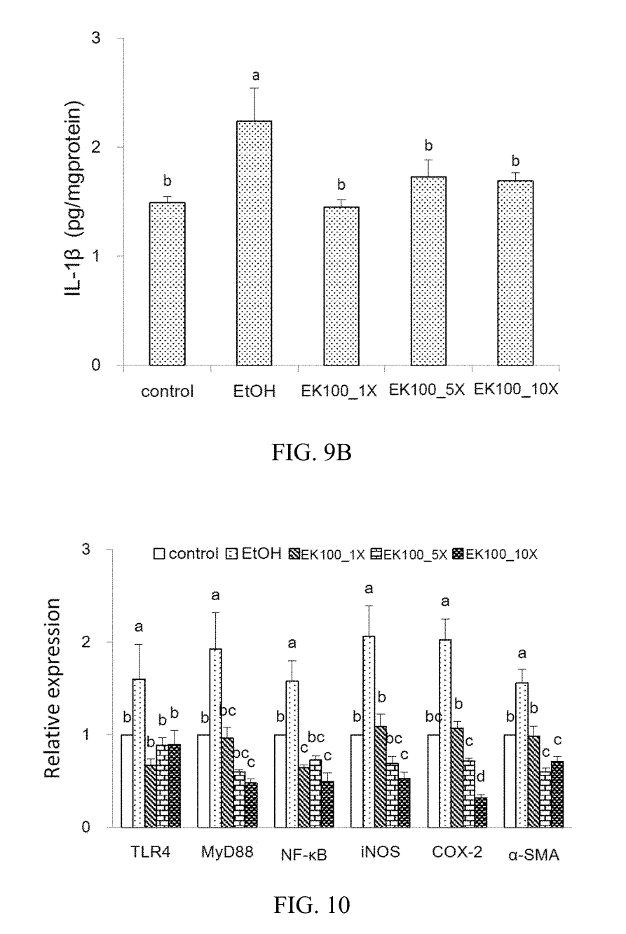

Mouse liver homogenates of each group provided in [Preparation Examples] D-3 were taken and diluted with Calibrator Diluent RD 5-17 to ⅓ of the original concentration respectively, and then a Quantikine® Rat TNF-α or a Quantikine® Rat IL-1β commercial kit (purchased from R&D Systems company, United States) was used for the Enzyme-linked immune-sorption assay (ELISA) to measure the amounts of tumor necrosis factor-alpha (TNF-α) and interleukin-1β (IL-1β) therein. The operation steps of Enzyme-linked immune-sorption assay were as follows: (i) monoclonal antibody with specificity to TNF-α (or IL-1β) was coated on each well of a 96-well plate; (ii) 50 μl of Assay Diluent RD1-41 was added into each well of the 96-well plate; (iii) 50 μl of standard or diluted liver homogenates with different concentrations (0, 12.5, 25, 50, 100, 200, 400, and 800 pg/ml) were added into different wells of the 96-well plate respectively, and then the plate was left standing at room temperature for 2 hours; (iv) each well was washed 5 times with 400 μl of wash buffer respectively; (v) the wash buffer was removed from each well completely, 100 μl of Rat TNF-α Conjugate (or Rat IL-1(3 Conjugate) was added into each well respectively, and then the plate was left standing at room temperature for 2 hours; (vi) each well was washed 5 times with 400 μl of wash buffer respectively; (vii) 100 μl of substrate solution was added into each well respectively, and then the plate was left standing in the dark at room temperature for 30 minutes; (viii) 100 μl of stop solution was added into each well respectively to stop the reaction; (ix) an ELISA hybrid reader (model number: Gen 5, purchased from BioTek company, England) was used to detect the absorbance of samples in each well at a wavelength of 450 nm, and the absorbances were then converted into the concentrations of TNF-α (or IL-1β). The results are shown in FIGS. 9A and 9B, wherein if the results of different groups are significantly different from each other (P value smaller than 0.05), those results are represented by different letters.

As shown in FIGS. 9A and 9B, as compared to the “control” mice, the concentrations of TNF-α and IL-1β in the liver of the “EtOH” mice were all significantly increased (p<0.05); however, as compared to the “EtOH” mice, the concentrations of TNF-α and IL-1β in the liver of the “EK100_1X,” “EK100_5X,” and “EK100_10X” mice were significantly decreased (p<0.05), or even decreased to that of the “control” mice. The above results indicate that alcohol consumption can induce liver inflammation, while ergostatrien-3β-ol is effective in alleviating the above inflammatory phenomenon.

5-2. Expressions of Pro-Inflammatory Genes

It has been known that TLR4, MyD88, NF-κB, iNOS, COX-2, α-SMA, etc. all belong to pro-inflammatory genes; thus, the expressions of the above genes in the mouse liver of each group were compared by the analysis method of [Preparation Examples] D-2. The results are shown in FIG. 10, wherein if the results of different groups are significantly different from each other (P value smaller than 0.05), those results are represented by different letters. As shown in FIG. 10, as compared to the “control” mice, the expressions of pro-inflammatory genes (i.e., TLR4, MyD88, NF-κB, iNOS, COX-2, and α-SMA) in the liver of the “EtOH” mice were all significantly increased (p<0.05); however, as compared to the “EtOH” mice, the expressions of pro-inflammatory genes in the liver of the “EK100_1X,” “EK100_5X,” and “EK100_10X” mice were significantly decreased (p<0.05), or decreased to that of or lower than that of the “control” mice. The above results indicate again that alcohol consumption can induce liver inflammation, while ergostatrien-3β-ol is effective in alleviating, inhibiting and/or treating the liver inflammation caused by alcohol consumption.

5-3. Hepatitis Histological Activity Index

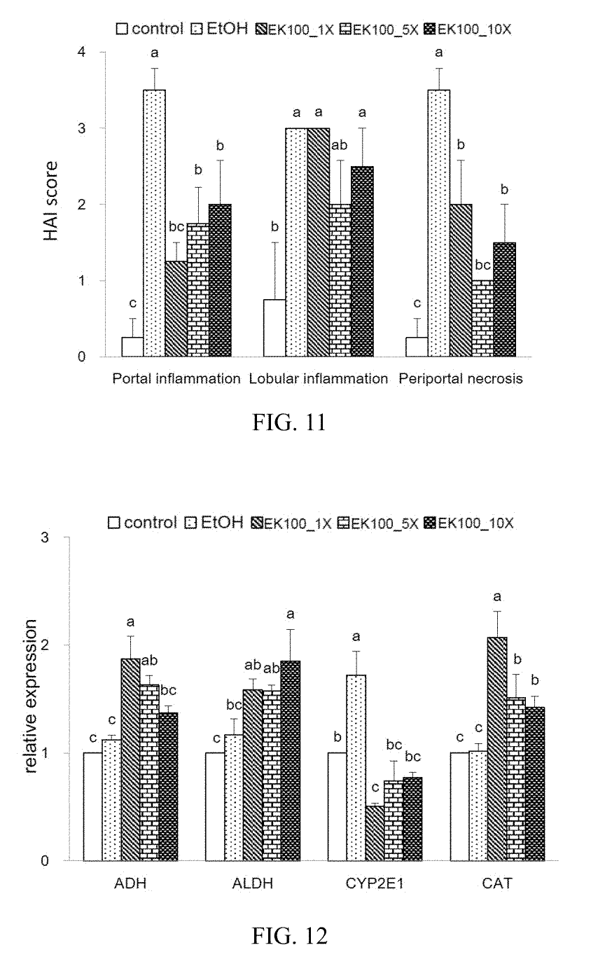

To further observe the inflammation of mouse liver, the internationally accepted hepatitis histological activity index (HAI score) was used to categorize the hepatitis activity into the portal zone, lobuli hepatis zone, and periportal zone, which were scored with 0 to 10 points (as shown in Table 8). The results are shown in FIG. 11, wherein if the results of different groups are significantly different from each other (P value smaller than 0.05), those results are represented by different letters. The above scoring method can be seen in articles, such as “Liver biopsy interpretation in chronic hepatitis. J Insur Med. 33: 110-113 (2001),” which is incorporated herein for reference.

TABLE 8

| Zone | Level of inflammation | Score |
|---|---|---|
| Portal | No portal inflammation | 0 |
| | Mild portal inflammation (inflammatory zone <1/3) | 1 |
| | Moderate portal inflammation (inflammatory zone <1/3) | 3 |
| | Severe portal inflammation (inflammatory zone <1/3) | 4 |
| Lob- | No lobular inflammation | 0 |
| ular | Mild lobular inflammation (inflammatory zone <1/3) | 1 |
| | Moderate lobular inflammation (inflammatory zone <1/3) | 3 |
| | Severe lobular inflammation (inflammatory zone <1/3) | 4 |
| Peri- | No periportal inflammation | 0 |
| portal | Mild piecemeal necrosis (smaller than 10% of | 1 |
| | periportal) | |
| | Moderate piecemeal necrosis (smaller than 50% of | 3 |
| | periportal) | |
| | Severe piecemeal necrosis (greater than 50% of | 4 |
| | periportal) | |
| | Moderate piecemeal necrosis plus bridging necrosis | 5 |
| | Severe piecemeal necrosis plus bridging necrosis | 6 |
| | Multilobular necrosis | 10 |

As shown in FIG. 11, as compared to the “control” mice, the necrosis scores for portal inflammation, lobular inflammation, and periportal inflammation of the liver of the “EtOH” mice were significantly increased (p<0.05); however, as compared to the “EtOH” mice, the necrosis scores of portal inflammation and periportal inflammation of the liver of the “EK100_1X,” “EK100_5X,” and “EK100_10X” mice were significantly decreased (p<0.05). The above results indicate again that alcohol consumption can induce liver inflammation, while ergostatrien-3β-ol is effective in alleviating, inhibiting and/or treating the liver inflammation caused by alcohol consumption.

### Example 6: Analysis of Alcohol Metabolism Capability of Mouse Liver

It has been known that alcohol dehydrogenase (ADH), acetaldehyde dehydrogenase (ALDH), catalase (CAT), and cytochrome (Cytochrome P450 2E1, CYP2E1) are the major enzymes of alcohol metabolism in the liver, wherein the CYP2E1 produces a great amount of ROS during the metabolic process, which is one of the reasons for the deterioration of alcoholic liver diseases. To understand whether ergostatrien-3β-ol could increase the alcohol metabolism capability of the liver, analysis was made of the gene expressions and protein expressions of CAT and CYP2E1, the gene expressions and activities of ADH and ALDH, and the alcohol concentrations in mouse serum.

6-1. Gene Expressions of Alcohol Metabolism-Related Enzymes

The expressions of alcohol metabolism-related enzymes (i.e., ADH, ALDH, CYP2E1, and CAT) in mouse liver of each group were compared by the analysis method of [Preparation Examples] D-2. The results are shown in FIG. 12, wherein if the results of different groups are significantly different from each other (P value smaller than 0.05), those results are represented by different letters. As shown in FIG. 12, as compared to the “control” mice, the gene expression of CYP2E1 in the liver of the “EtOH” mice was significantly increased (p<0.05); however, as compared to the “EtOH” mice, the gene expressions of CYP2E1 in the liver of the “EK100_1X,” “EK100_5X,” and “EK100_10X” mice were significantly decreased (p<0.05), or even decreased to that of the “control” mice. On the other hand, as compared to the “EtOH” mice, the gene expressions of ADH, ALDH, and CAT in the liver of the “EK100_1X,” “EK100_5X,” and “EK100_10X” mice were significantly increased (p<0.05). The above results indicate that alcohol consumption can decrease the alcohol metabolism capability of the liver, while ergostatrien-3β-ol is effective in increasing the alcohol metabolism capability of the liver.

6-2. Protein Expressions of Alcohol Metabolism-Related Enzymes

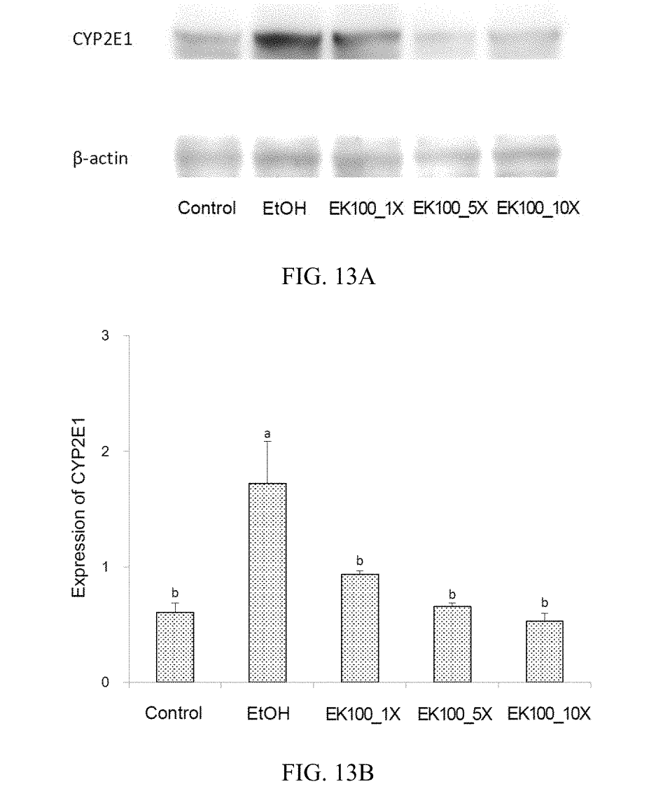

The protein expressions of alcohol metabolism-related enzymes (e.g., CYP2E1) in mouse liver homogenates of each group provided in [Preparation Examples] D-3 were compared by western blot. The operation steps were as follows: (i) each liver homogenate was mixed with stain at a ratio of 1:4, respectively; (ii) the mixture was heated at 95° C. for 5 minutes, and then loaded into a sodium dodecyl sulphatepolyacrylamide gel (SDS-PAGE) with a concentration of 12% in running gel and a concentration of 5% in stacking gel; (iii) 70 volts was applied to perform the electrophoresis until all of the samples in each lane reached the top of the running gel, and then 100 volts was applied to continue the electrophoresis; (iv) after the electrophoresis was completed, the proteins on the SDS-PAGE were transferred to a PVDF film by semi-dry transfer process (10 volts, 10 minutes); (v) after the transference was completed, 2.5% bovine serum albumin (BSA) was added on the PVDF film, and then the PVDF film was kept at 4° C. overnight; (vi) a primary antibody that was diluted in an appropriate ratio was added on the PVDF film, and then the PVDF film was kept at 4° C. overnight; (vii) the PVDF film was washed 4 times with washing buffer, each for 5 minutes; (viii) a secondary antibody was added on the PVDF film, and then the PVDF film was kept at room temperature for 2 hours; (ix) the PVDF film was washed 4 times with washing buffer, each for 5 minutes; (x) the protein expression of CYP2E1 was detected by the enhanced chemiluminescence (ECL), photographed and then analyzed quantitatively. The photographs are shown in FIG. 13A, and the results of quantitation are shown in FIG. 13B, wherein if the results of different groups are significantly different from each other (P value smaller than 0.05), those results are represented by different letters.

As shown in FIGS. 13A and 13B, as compared to the “control” mice, the protein expression of CYP2E1 in the liver of the “EtOH” mice was significantly increased (p<0.05); however, as compared to the “EtOH” mice, the protein expressions of CYP2E1 in the liver of the “EK100_1X,” “EK100_5X,” and “EK100_10X” mice were significantly decreased (p<0.05). The above results indicate again that alcohol consumption can decrease the alcohol metabolism capability of the liver, while ergostatrien-3β-ol is effective in increasing the alcohol metabolism capability of the liver.

6-3. Activities of Alcohol Metabolism-Related Enzymes

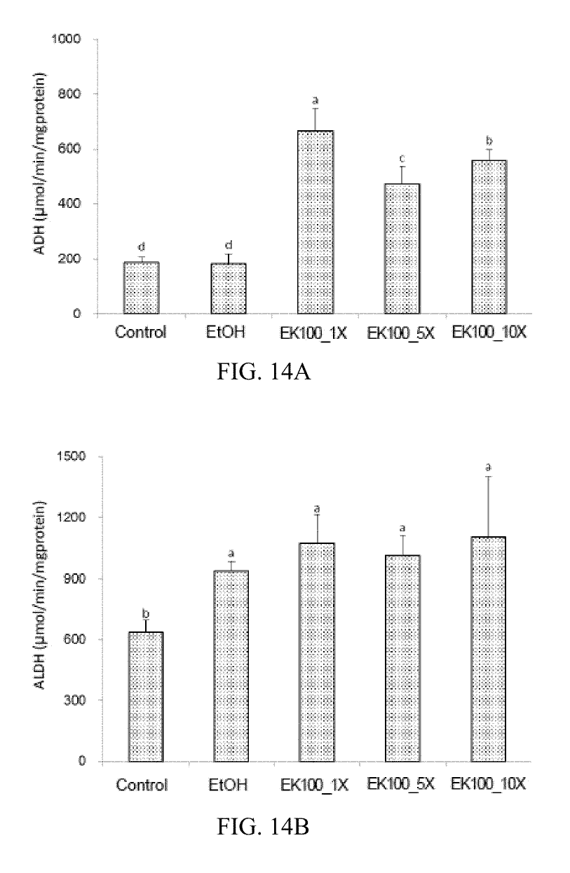

The activities of ADH in mouse liver homogenates of each group provided in [Preparation Examples] D-3 were analyzed. The operation steps were as follows: (i) 50 μl of liver homogenate, 50 μl of phosphate-buffered saline (PBS), and 1 ml of 0.1 M glycine-NaOH buffer (pH 10.8, 10 mM NAD⁺) were well mixed; the mixture was left standing in the dark at room temperature for 4 minutes; and then the absorbance (A) of the mixture was measured at 340 nm wavelength; (ii) 50 μl of liver homogenate, 50 μl of phosphate-buffered saline (PBS), and 1 ml of 0.1 M glycine-NaOH buffer (pH 10.8, 10 mM NAD⁺, 0.016 M ethanol) were well mixed; the mixture was left standing in the dark at room temperature for 4 minutes; and then the absorbance (B) of the mixture was measured at 340 nm wavelength; (iii) absorbance (A) and absorbance (B) were substituted into formula 2 below to calculate the activity of ADH (μmole/min/mg protein). The results are shown in FIG. 14A, wherein if the results of different groups are significantly different from each other (P value smaller than 0.05), those results are represented by different letters.

$$activity of ADH = \frac{( B - A ) \times 2 \times 100}{\begin{matrix}0.622 \times 4 mins \times 0.01 \times \\ concentration of liver protein\end{matrix} } \qquad Formula 2$$

Furthermore, the activities of ALDH in mouse liver homogenates of each group provided in [Preparation Examples] D-3 were analyzed. The operation steps were as follows: (i) 100 μl of liver homogenate and 1 ml of sodium pyrophosphate buffer (pH 8.8, 1 mM NAD⁺, 0.2 mM 4-methylpyrazole, 1 mM MgCl₂, 2 μM rotenone, 1% Triton X-100) were well mixed; the mixture was left standing in the dark at room temperature for 20 minutes; and then the absorbance (A) of the mixture was measured at 340 nm wavelength; (ii) 100 μl of liver homogenate and 1 ml of sodium pyrophosphate buffer (pH 8.8, 1 mM NAD⁺, 0.2 mM 4-methylpyrazole, 1 mM MgCl₂, 2 μM rotenone, 1% Triton X-100, 5 mM acetaldehyde) were well mixed; the mixture was left standing in the dark at room temperature for 20 minutes, and then the absorbance (B) of the mixture was measured at 340 nm wavelength; (iii) absorbance (A) and absorbance (B) were substituted into formula 3 below to calculate the activity of ALDH (μmole/min/mg protein). The results are shown in FIG. 14B, wherein if the results of different groups are significantly different from each other (P value smaller than 0.05), those results are represented by different letters.

$$activity of ALDH = \frac{( B - A ) \times 100}{\begin{matrix}0.622 \times 20 mins \times 0.01 \times \\ concentration of liver protein\end{matrix} } \qquad Formula 3$$

As shown in FIG. 14A, compared to the “control” mice or the “EtOH” mice, the activities of ADH in the liver of the “EK100_1X,” “EK100_5X,” and “EK100_10X” mice were all significantly increased (p<0.05). As shown in FIG. 14B, compared to the “control” mice, the activity of ALDH in the liver of the “EK100_1X,” “EK100_5X,” and “EK100_10X” mice were all significantly increased (p<0.05). The above results indicate again that ergostatrien-3β-ol is effective in increasing the alcohol metabolism capability of the liver.

6-4. Alcohol Concentration in Mouse Serum

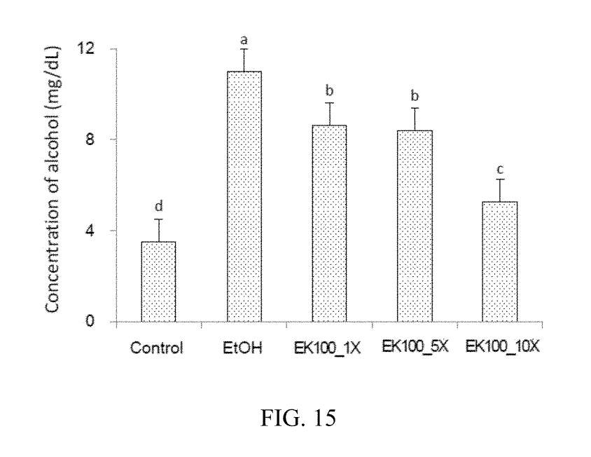

Mouse serum samples of each group provided in [Preparation Examples] C-2 were well mixed with ADH and NAD⁺ reagents in a commercial kit (Dade-Berhring company, England, Emit® II Plus Ethyl alcohol assay), and then the alcohol concentrations in the above serum samples were measured by an automatic biochemistry analyzer (Olympus company, Japan, AU2700) at 340 nm wavelength. The results are shown in FIG. 15, wherein if the results of different groups are significantly different from each other (P value smaller than 0.05), those results are represented by different letters.

As shown in FIG. 15, as compared to the “control” mice, the alcohol concentration in the serum of the “EtOH” mice was significantly increased (p<0.05); however, as compared to the “EtOH” mice, the alcohol concentrations in the serum of the “EK100_1X,” “EK100_5X,” and “EK100_10X” mice were significantly decreased (p<0.05). The above results indicate again that alcohol consumption can decrease the alcohol metabolism capability of the liver, while ergostatrien-3β-ol is effective in increasing the alcohol metabolism capability of the liver.

As shown in the above experiments, ergostatrien-3β-ol is effective in increasing the alcohol metabolism capability of the liver. For a subject with body fat accumulation or hepatic injuries caused by alcohol consumption, ergostatrien-3β-ol is effective in alleviating and/or inhibiting body fat accumulation (e.g., liver fat accumulation), or alleviating, inhibiting, and/or treating the hepatic injuries.

What is claimed is:

1. A method of alleviating, inhibiting and/or treating hepatic injuries caused by alcohol consumption, comprising administering to a subject in need an effective amount of an active ingredient selected from the group consisting of ergstatrien-3β-ol, a pharmaceutically acceptable ester of ergstatrien-3β-ol, and combinations thereof.

2. The method as claimed in claim 1, wherein the alcohol consumption is chronic alcohol consumption.

3. The method as claimed in claim 1, wherein the hepatic injuries is at least one of hepatitis, liver fibrosis, liver cirrhosis, and hepatic carcinoma.

4. The method as claimed in claim 2, wherein the hepatic injuries is at least one of hepatitis, liver fibrosis, liver cirrhosis, and hepatic carcinoma.

5. The method as claimed in claim 1, wherein the active ingredient is administered at an amount ranging from about 1 mg (as ergstatrien-3β-ol)/kg-body weight to about 100 mg (as ergstatrien-3β-ol)/kg-body weight per day.

6. The method as claimed in claim 1, wherein the active ingredient is administered at an amount of about 10 mg (as ergstatrien-3β-ol)/kg-body weight per day.

7. A method of alleviating and/or inhibiting body fat accumulation caused by alcohol consumption, comprising administering to a subject in need an effective amount of an active ingredient selected from the group consisting of ergstatrien-3β-ol, a pharmaceutically acceptable ester of ergstatrien-3β-ol, and combinations thereof.

8. The method as claimed in claim 7, wherein the body fat accumulation is liver fat accumulation.

9. The method as claimed in claim 7, wherein the body fat accumulation is caused by chronic alcohol consumption.

10. The method as claimed in claim 8, wherein the liver fat accumulation is caused by chronic alcohol consumption.

11. The method as claimed in claim 7, wherein the active ingredient is administered at an amount ranging from about 1 mg (as ergstatrien-3β-ol)/kg-body weight to about 100 mg (as ergstatrien-3β-ol)/kg-body weight per day.

12. The method as claimed in claim 7, wherein the active ingredient is administered at an amount of about 10 mg (as ergstatrien-3β-ol)/kg-body weight per day.

13. A method of increasing the alcohol metabolism capability of the liver, comprising administering to a subject in need an effective amount of an active ingredient selected from the group consisting of ergstatrien-3β-ol, a pharmaceutically acceptable ester of ergstatrien-3β-ol, and combinations thereof.

14. The method as claimed in claim 13, wherein the active ingredient is administered at an amount ranging from about 1 mg (as ergstatrien-3β-ol)/kg-body weight to about 100 mg (as ergstatrien-3β-ol)/kg-body weight per day.

15. The method as claimed in claim 13, wherein the active ingredient is administered at an amount of about 10 mg (as ergstatrien-3β-ol)/kg-body weight per day.

16. The method as claimed in claim 13, wherein the active ingredient is administered prior to, during the course of, and/or after alcohol consumption.
